(19) United States
(12) Patent Application Publication
(10) Pub. No.: US 2023/0211362 A1
(43) Pub. Date: Jul. 6, 2023

(54) A METHOD OF COATING AN OCULAR PROSTHETIC DEVICE AND OTHER PROSTHETICS

(71) Applicant: UEA Enterprises Limited, Norwich Norfolk (GB)

(72) Inventors: Sheng QI, Norwich Norfolk (GB); Julie SANDERSON, Norwich Norfolk (GB); Matthew ALEXANDER, Norwich Norfolk (GB)

(21) Appl. No.: 17/928,037

(22) PCT Filed: May 28, 2021
(86) PCT No.: PCT/GB2021/051334

§ 371 Date: Nov. 28, 2022

(30) Foreign Application Priority Data
May 29, 2020 (GB) 2008098.2

(51) Int. Cl. B05B 5/025; A61L 27/34; A61L 27/54

(52) U.S. Cl. CPC B05B 5/0255; A61L 27/34; A61L 27/54

(57) ABSTRACT

Described herein is a method of coating a prosthetic device, such as an ocular prosthetic device, the method comprising nanoelectrospraying droplets comprising an active ingredient and/or a carrier species onto a surface of the prosthetic device in a predetermined pattern, the nanoelectrospraying involving controlling the flow rate of the droplets from a nozzle of the nanoelectrospraying equipment by controlling the voltage between the nozzle and the ocular prosthetic device. Also described herein are ocular prosthetic device formable according to the method.

There is a wide range of applications of using contact lens (CLs) as carriers to deliver and release compounds to the eye. These reported methods can be divided into 5 main categories [12, 13]: 1) drug soaking, where the lenses are typically soaked in drug solution for a period of time 2) molecular imprinting, in which a hydrogel polymer is synthesised as a complimentary template for the drug molecule to give drug binding sites within the polymer 3) layering, where typically a drug-polymer is manufactured in a layer within the lens [e.g. see U.S. Pat. No. 6,719,929 B2] 4) chemically treat and modify the lens surface [e.g. see EP 1 315 985 B1; EP 1 046 068 B1; EP 1 299 753 B1] and 5) additive coating onto the lenses by electrospinning [US 2004/0170666 A1; U.S. Pat. No. 9,956,168 B2], electrospray [US 2007/0157880 A1 EP 2 529 761 B1; EP 1988941]; or inkjet printing [EP2555751A1; US201816188833].

The drug soaking method relies on passive diffusion of a drug into the lens materials. This process is simple, but wasteful. The capacity is low and loading and release from soaked lenses are highly uncontrolled leading to rapid release kinetics and a varying rate of release. The molecular imprinting method allows the incorporation of drug molecules into the contact lenses polymeric material, although typically only of one drug at a time. This drug loading method could extend the release of drug to weeks. However due to differences in the chemistry of each ocular drug, each contact lens material would be different, so regulatory approval would be needed for each different drug to reach the market. Using the layering technique, the manufacture process contains multiple steps and is highly complex. For example, latanoprost DECLs that have been made in this way have an encapsulated drug-polymer film within the contact lens hydrogel [14]. The drug-polymer film has to be placed within the contact lens hydrogel and the dried hydrogel then has to be precisely lathed so that the drug-polymer is encapsulated by the contact lens hydrogel.

Additive coating contact lenses using drug-containing materials has also been proposed. The coating concept is simple and has the advantage of potentially being an additive step that can be implemented into the manufacturing process of the contact lenses. Spin coating, which involve centrifugal coverage of the entire lens, has been suggested and previously tested by us as a coating technique but suffers from the disadvantages of high drug wastage as the drug-polymer is lost from the edge of the lenses, which leads to a low level of drug loading accuracy. In addition, if the drug-loaded film has any opacity after hydration, the lenses will lose their clarity and affect vision. Conventional electrospinning and electrospray [e.g. see US 2004/0170666 A1; U.S. Pat. No. 9,956,168 B2, US 2005/0067287 A1; US 2007/0157880 A1; EP 2 529 761 E1; EP 1988941] have also been reported for coating of hydrogel surface and contact lenses.

The difference between the invention described in this disclosure and the above-mentioned reference is that conventional electrospray and electrospinning require the pumping mechanism (i.e. syringe pumps) to continuously feed the solution to the nozzle for deposition, as illustrated in FIG. 2. Additionally, conventional electrospray and electrospinning techniques use a constant voltage. For these systems, they are not operated on the principle of drop-on-demand and the accuracy and consistence of control of the flow rate by the pumps can affect the quality of the liquid deposition. The conventional electrospinning and electrospraying generate a layers of nanoparticles or nanofibers coat on the hydrogel surfaces, have no selectivity on the location of the material deposition. Therefore, the coating is non-selective and provides the coverage even wider than the entire lens. This leads to two significant disadvantages: 1) waste of active ingredient and no control of the amount of the materials that are deposited on the lens; 2) the full coverage of the lens surface leads to the loss of clarity of vision zone of lens, as shown in the FIG. 3.

Inkjet printing is an established drop-on-demand technique that can deposit low viscosity liquid formulations (<30 cp) with typical drop volumes of 1-5 pL and a resulting feature size of 20-50 micron [16]. Although the inkjet method may be used to selectively print on certain portions of the lens surface, it will suffer significant difficulties in coating the hemispherical geometry of a contact lens due to highly constrained printable liquid properties. Inkjet requires low-viscosity, high-solvent content which would lead to long drying times for printed material and issues of flow and spreading of the drug coating post-deposition due to the curved substrate. This could lead to unwanted coating within the visual axis of the lens. Another drawback of inkjet is that the low printing resolution results in a relatively large droplet volume which will produce a sudden step at the edge of the deposited drug film. This relatively prominent feature could be problematic and cause increased friction due to blinking and discomfort to the wearer.

The present inventors wished to develop a method that is an alternative to, ideally an improvement upon, the various prior art methods of applying an active compound to a contact lens or other ocular prosthetic device, and which addresses one or more of the problems associated with the prior art techniques.

### SUMMARY OF THE INVENTION

In a first aspect, there is provided a method of coating an ocular prosthetic device, the method comprising

nanoelectrospraying droplets comprising an active ingredient and/or a carrier species onto a surface of the ocular prosthetic device in a predetermined pattern, the nanoelectrospraying involving controlling the flow rate of the droplets from a nozzle of the nanoelectrospraying equipment by controlling the voltage between the nozzle and the ocular prosthetic device.

In a second aspect, there is provided an ocular prosthetic device formable according to the method of the first aspect. Any liquid in the droplets will typically have been removed (e.g. evaporated) to leave a layer comprising the active ingredient and/or carrier species on the surface of the prosthetic device.

In a third aspect, there is provided a method of coating a surface of a prosthetic device, the method comprising

nanoelectrospraying droplets comprising an active ingredient and/or a carrier species onto the surface in a predetermined pattern, the nanoelectrospraying involving controlling the flow rate of the droplets from a nozzle of the nanoelectrospraying equipment by controlling the voltage between the nozzle and the prosthetic device. The surface may be of any curvature, e.g. curved (which may be selected from convex and concave) or flat. The surface may be electrically conducting, insulating or electrically semi-conducting. Preferably, the surface is a curved and/or electrically conductive surface.

In a fourth aspect, there is provided a prosthetic device formable according to the method of the third aspect. The surface onto which the droplets have been nanoelectrosprayed may be of any curvature, e.g. curved or flat. The surface onto which the droplets have been nanoelectrosprayed may be electrically conducting, insulating or electrically semi-conducting. The prosthetic device may be a device with a curved and/or electrically conductive surface (and which is the surface on with the droplets are nanoelectrosprayed), such as a stent. Any liquid in the droplets will typically have been removed (e.g. evaporated) to leave a layer comprising the active ingredient and/or carrier species on the surface of the prosthetic device.

The present invention is a unique liquid deposition method involving nanoelectrospray (nES) onto ocular and other prosthetic devices, which allows high precision control to deposit controlled mass of materials onto surfaces, e.g. electrically conductive surfaces, of the ocular prosthetic device or other prosthetic device with pre-designed pattern at pre-determined location. In more detail, the method allows precise deposition of a desired amount of material onto a surface, which may be an electrically conductive surface (such as a wet surface or a metal surface), to form a coating layer on the surface. This allows the formation of the pre-designed coating pattern of the materials that have been deposited onto the conductive surface. The coat material and the pattern in combination carry functionality such as controlled delivery of drug at specific site of the body. Examples of such include a ring-shaped drug layer at the edge of contact lenses to allow targeted drug delivery to the eye at a controlled rate. It may also be applied to other prosthetics, e.g. by applying a drug-containing coating onto the highly curved surfaces of carotid artery stents. Depending on the processing parameters and the material properties, the deposited coat layer can be either a smooth and continuous layer of materials with no pinhole or a layer formed by semi-fused nanoparticles. The method can also generate multiple-layer deposition in which the layers overlay with each other. The dimension of the area covered by the deposition is tuneable by the nES operational parameters.

The method has distinct difference from conventional electrospray method in terms of the method used to control the liquid jetting for deposition. This leads to much narrower and streamlined spray of the material leading to more precise and selective deposition location of the materials in comparison to conventional electrospray. It does not allow deposition of a material onto a large non-selective area.

The advantages can include:
enabling the high precision droplet deposition and allowing for patterned printing and improved drug-dosing accuracy
operating in purely additive and drop-on-demand printing mode
being able to additive print drug formulations with high viscosity, for example much higher than liquid formulations usually used for ink-jet printing, e.g. up to 10000 cP viscosity
a very fast coating process—the coating process can be completed within a period of few seconds to tens of seconds (depending on the amount of materials required to be deposited). This short processing time-scale may therefore allow the process to be implemented as an additive manufacturing step into the contact lens production process.
the materials and the number of depositions are highly flexible. This gives the potential of the technology to be used for individualised/personalised medical product production.

As mentioned above, some prior art techniques have two significant disadvantages: 1) waste of active ingredient and no control of the amount of the materials that are deposited on the lens; 2) the full coverage of the lens surface leads to the loss of clarity of vision zone of lens, as shown in the FIG. 3 below. The method of the present invention overcomes both disadvantages by a selective, typically significantly reduced and tuneable, deposition area and the unique drop-on-demand liquid deposition control.

Herein we describe the use of additive deposition technology using nanoelectrospray (nES), to deposit the drug formulation on selected areas of an ocular prosthetic device, e.g. a contact lens. nES is a form of ES operation where the fluid flow rate is controlled by the applied voltage and nES is achieved without the need for pumps or valves [17, 18]. In nES, for a particular applied voltage, the droplet size and volumetric flow rate will depend on (as well as the applied voltage), the liquid properties such as conductivity, surface tension and viscosity and on nozzle geometry. It has been shown to be a versatile tool for numerous applications but most notably, it has been proven as a benign technique for the ‘soft’ ionisation of biomolecules, most commonly used in mass spectrometry. At present, the most studied ES form is the cone-jet regime, wherein the balance between electrostatic stresses and surface tension creates a Taylor cone and from the apex of which a liquid jet is emitted [17].

Conventional electrospray (ES) for additive printing can generate liquid droplets of microns to nanometer size by electrostatic force to form a film coat. However as demonstrated in FIG. 3, whilst ES work successfully proved the technical feasibility of material deposition, the coating of the entire inner surface of the lens causes vision interference if the coating is not completely transparent. nES can overcome the limitations of alternative techniques, including conventional ES, for the production of drug eluting contact lenses. The nES printing technique has been demonstrated for controlled ejection and deposition of minute volumes (sub-picolitre) of highly viscous liquid (up to 10000 cp) not possible by conventional inkjet printing. Due to the precise control of volumetric throughput and deposition rate onto the substrate, nES can not only allow the printing on wet contact lenses, but also produce shallow/smooth edge profile to increase patient comfort.

Therefore, nES can be used to coat the ocular prosthetic device, e.g. a contact lens, with a continuous coating in a certain desired pattern, e.g. to coat a fraction of the surface area of the ocular prosthetic device, e.g. a contact lens, allowing for active ingredient for delivery to the eye at from the coated area(s). nES can generate liquid droplets 10s of microns to nanometer size and with the use of appropriate formulation and processing parameters, the technique can deposit semi-dried droplets to form a film coat on the ocular prosthetic device's, e.g. a contact lens', surface. The film coat may contain excipients including a polymer and an active compound. This ability to print higher drug and polymer loadings with lower solvent content, when compared to inkjet, would result in minimal flow of deposited material prior to drying with the ability to design formulations to fuse or dry almost instantaneously on landing, of particular benefit when addressing wet contact lenses. Regarding patient comfort, due to the high-resolution and low-volume capabilities the film coating profile can be controlled to result in a shallower/smoother edge profile and increased patient comfort due to the deposition of nm to micron-sized droplets within the spray.

More importantly, instead of printing the formulation to cover the entire surface of the ocular prosthetic device, e.g. a contact lens, nES is capable of selectively coating a certain proportion of ocular prosthetic device's, e.g. a contact lens', surface to achieve the patterned printing (as seen in FIG. 4). nES deposition has high-precision in droplet deposition and can achieve patterned printing and highly controlled volume dispensing to improve the dosing accuracy. nES is a form of ES operation where the fluid flow rate is exclusively controlled by the applied voltage and nozzle geometry and nES can be achieved without the need for pumps or valves [8,9].

nES allows the controlled ejection and deposition of minute volumes (sub-picolitre) of highly viscous liquid (up to 1000 cp, required for drug formulation) which is not possible by conventional inkjet printing. The nES printing technique allows excellent reproducibility with volume variation demonstrated to be less than 3% to enable high precision drug dosing.

### BRIEF DESCRIPTION OF THE FIGURES

FIG. 1 shows molecular structures of some example drugs and polymers that may be applied using the method described herein.

FIG. 2 shows schematically the setup of a conventional electrospinning/electrospraying apparatus

FIG. 3 shows (a) conventional, i.e. prior art electrospray set up, e.g. as described by patent EP 2 529 761 B1; (b) electrospray coated contact lens with PLGA and timolol before and after drying; (c) SEM images of the cross-section and (d) surface of the nanoparticle-based coating layer. The tests using the prior art method were carried out by the present inventors.

FIG. 4 shows examples of patterns that can be printed onto the ocular prosthetic device, e.g. a contact lens, for the purpose of delivering single or multiple compounds.

FIG. 5 shows an example of the apparatus for carrying of an nES process for coating a contact lens.

FIG. 6. shows (a) CCD image of a nanoelectrospray printed array of 1600 replica drops of UV curable polymer (150 cps) with the mean dot diameter as 7.1 μm with standard deviation of 0.2 μm demonstrating the reproducibility and accuracy of the nanoelectrospray method; (b) CCD image of a nanoelectrospray printed continuous line demonstrating the printing capability.

FIG. 7 shows temporal changes in applied voltage waveform and spray current during nES Printing.

FIG. 8 shows the nES coating width with different nozzle substrate distance.

FIGS. 9(A) to (D) show a microscopic image of 1 layer (A) and 5 layers (B) of nES PLGA on glass substrate. Picture C and D showed the corresponding surface profile by the profilometer.

FIGS. 10(A) to (F) shows imaging of the hydrated soft contact lenses, immediately after nES with 30 rounds of PLCL/Ketotifen a), light transmission through a control lens b) and nES lens c) using a CCD camera presenting light transmission (black) and areas of opacity (white) around the periphery caused by polymer/drug deposition. Scanning electron microscope images depict a rough topography where the polymer/drug was deposited onto the lens. At ×41 magnification the polymer/drug is shown to be deposited as a band approximately 800 μm wide d). The contact lens has a typically smooth appearance with a continuous deposition of polymer and drug at ×99 magnification e). A cross-section at ×2000 magnification, reveals the thickness of the polymer/drug layer of approximately 4 μm f.

FIG. 11 shows In vitro drug release data of nES coats on contact lenses of (a) PLCL 70:30 with ketotifen; (b) 5% w/v Zein with ketotifen; and (c) 2.5% w/v Zein with ketotifen.

FIG. 12 (A1) shows an UV calibration curve for in vitro release of ketotifen in PBS pH 7.4.

FIG. 13 (A2) shows an UV calibration curve of ketotifen in the mixture of pH 7.4 PBS:Ethanol (1:1).

FIG. 14 (A3) shows an UV calibration curve of ketotifen in the mixture of DCM:Ethanol (1:1).

FIG. 15 (A5) shows a surface profile (measured by surface profilometer) of a nanoelectrosprayed line of PLGAₐ (see Examples for more details on conditions relating to PLGAₐ to PLGA_{g})

FIG. 16 (A6) shows a surface profile (measured by surface profilometer) of a nanoelectrosprayed line of PLGA_{b}

FIG. 17 (A7) shows a surface profile (measured by surface profilometer) of a nanoelectrosprayed line of PLGA_{c}

FIG. 18 (A8) shows a surface profile (measured by surface profilometer) of a nanoelectrosprayed line of PLGA_{d}

FIG. 19 (A9) shows a surface profile (measured by surface profilometer) of a nanoelectrosprayed line of PLGAₑ

FIG. 20 (A10) shows a surface profile (measured by surface profilometer) of a nanoelectrosprayed line of PLGA_{f}

FIG. 21 (A11) shows a surface profile (measured by surface profilometer) of a nanoelectrosprayed line of PLGA_{g}

FIG. 22 (A12) shows a microscopic image (top-down view) of a nanoelectrosprayed line of PLGA_{d}

FIG. 23 (A13) shows a microscopic image (top-down view) of a nanoelectrosprayed line of PLGAₑ

FIG. 24 (A14) shows a microscopic image (top-down view) of a nanoelectrosprayed line of PLGA_{f}

FIG. 25 (A15) shows a microscopic image (top-down view) of a nanoelectrosprayed line of PLGA_{g}

FIG. 26 shows the sprayed zein rings on a glass substrate in triplicate with different levels (left) and the locations of measurement of the zein film by a contact profilometer (right).

FIG. 27 shows the surface profile and a digital image of a thin film deposited on a glass slide with 2.5% w/v zein: zein A (a), zein B (b), 5% w/v zein: zein C (c) and zein D (d).

FIG. 28 shows the spraying width and step height of zein B (2.5% w/v) and zein D (5% w/v) with different spraying parameters.

FIG. 29 shows the targeted deposition of a polymer-drug layer on a contact lens by nanoelectrospraying (A), a 3D model of the lens holder (B), a 3D-printed lens holder containing a blank contact lens (C) and an the illustration of the nozzle to substrate distance (D).

FIG. 30 contains digital photographs of a contact lens before (A) and directly after nanoelectrospraying drug-polymer compositions onto the contact lens (B: zein+BIM, C: zein+KF).

FIG. 31 shows cryo-SEM images of the nanoelectrosprayed Zein+BIM (A: cross-section, B: surface) and Zein+KF (C: cross-section, D: surface) on contact lenses.

FIG. 32 shows the cumulative drug release of ketotifen fumarate (top) and bimatoprost (bottom) from contact lenses prepared by nanoelectrospraying.

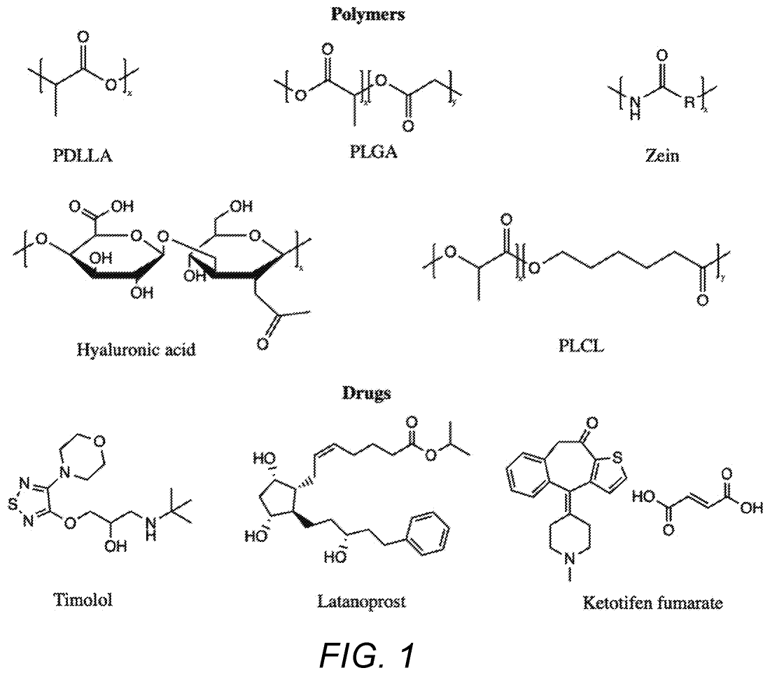

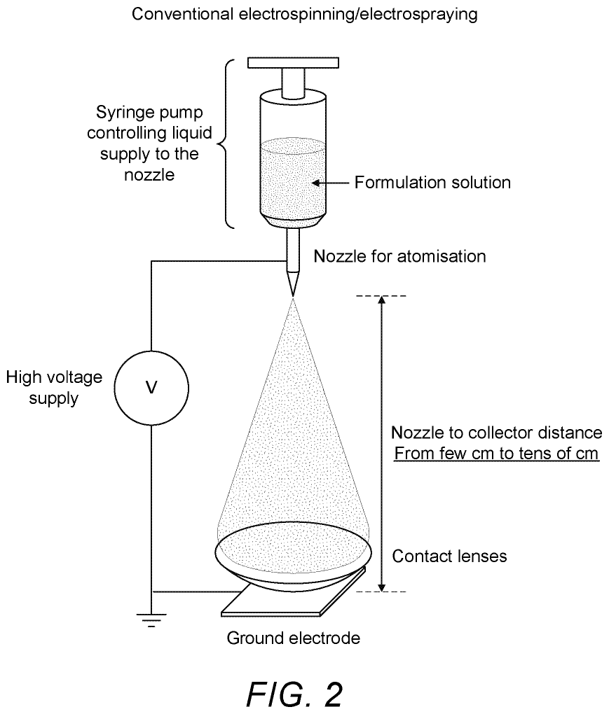

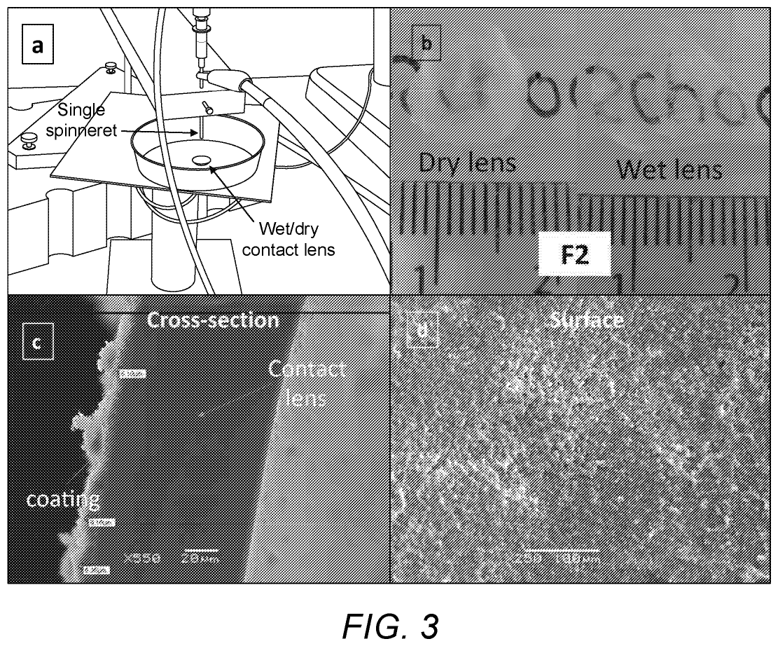

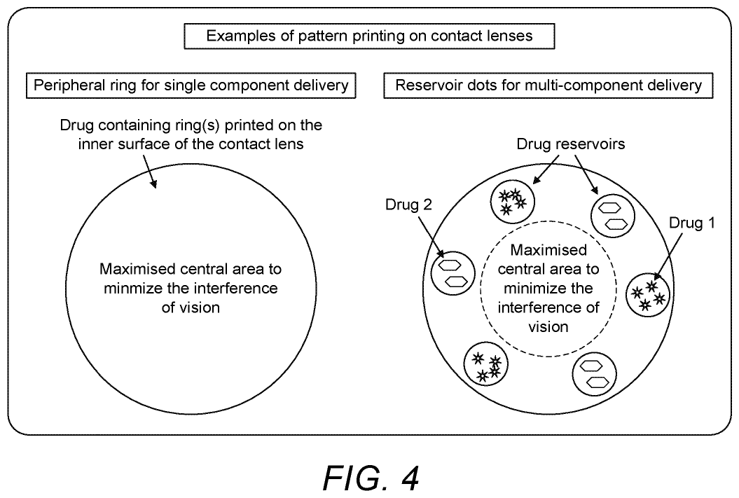

### DETAILED DESCRIPTION

In a first aspect, there is provided a method of coating an ocular prosthetic device, the method comprising

nanoelectrospraying droplets comprising an active ingredient and/or a carrier species onto the surface of the ocular prosthetic device in a predetermined pattern, the nanoelectrospraying involving controlling the flow rate of the droplets from a nozzle of the nanoelectrospraying equipment by controlling the voltage between the nozzle and the ocular prosthetic device.

Nanoelectrospraying involves controlling the flow rate of the droplets from a nozzle of the nanoelectrospraying equipment by controlling the voltage between the nozzle and the ocular prosthetic device. The ocular prosthetic device may be placed on a surface, e.g. an electrically conductive surface. The electrically conductive surface may comprise an electrically conductive material, which may be selected from a metal, carbon, and organic or inorganic electrically conducting material. The metal may be any metal, including any transition metal, such as, for example, copper, brass, zinc, iron, steel, gold, silver. The surface may comprise a transparent, electrically conducting material, such as a transparent conducting oxide, which may be, for example, selected from an indium tin oxide, which may be doped, for example with fluorine. doped zinc oxide, such as indium zinc oxide, aluminium zinc oxide. The organic electrically conducting material may comprise an electrically conductive polymer, for example a polymer selected from poly(3,4-ethylenedioxythiophene) (PEDOT), poly(3,4-ethylenedioxythiophene) PEDOT: poly(styrene sulfonate) PSS (i.e. the material PEDOT:PSS), poly(4,4-dioctyl cyclopentadithiophene). The carbon may be any form of electrically conducting carbon, which may comprise, for example graphite. In an embodiment, the carbon comprises a carbon nanotube, and the surface comprising the carbon nanotube may be transparent. An advantage of the surface being transparent is that it allows light transmission from below the ocular prosthetic device, which is typically transparent, which allows more accurate analysis of the ocular prosthetic device and any materials applied to it during and after coating process.

Controlling the flow rate of droplets may be carried out by setting a nozzle to substrate distance (the substrate being the ocular prosthetic device), and then determining the minimum (onset) voltage between the nozzle and the ocular prosthetic device for a droplet to eject from the nozzle (Vₒₙ). To eject droplets from the nozzle, the voltage may then be altered from a voltage V₁, which is below Vₒₙ, to V₂, which is above Vₒₙ. V₁, may be ground (0V) or a voltage less than Vₒₙ. V₂-V₁ may be of from about 0.05 kV to 5 kV, optionally 0.05 kV to 4 kV, optionally 0.05 kV to 3 kV, optionally 0.05 kV to 2 kV, optionally 0.05 kV to 1 kV, optionally 0.05 kV to 0.5 kV, optionally about 0.1 to 0.4 kV. V₂ will depend on a variety of factors, including the nature of the droplets and the distance from nozzle to substrate. However, in general terms, V₂ may be from 1.5 kV to 3 kV. The voltage may be circulated between V₁ and V₂ with a particular frequency. The frequency may be from 0.1 Hz to 20 kHz, optionally 0.5 Hz to 15 kHz, optionally 1 Hz to 10 kHz, optionally optionally 1 Hz to 5 kHz, optionally 1 Hz to 1 kHz, preferably optionally 10 Hz to 1000 Hz is preferably 1 to 20 Hz, preferably 5 to 15 Hz. While the nozzle is not spraying (e.g. when the voltage is at V₁), the ocular prosthetic device may be moved relative to the nozzle, and then the ocular prosthetic device held stationery relative to the nozzle while spraying, and this may be repeated, to allow a build-up of a pattern on the surface. In alternative embodiments, there may be movement of the ocular prosthetic device during spraying, to allow build-up of a pattern.

Controlling the voltage between the nozzle and the ocular prosthetic device may mean varying the voltage to induce droplet formation. In some examples, varying the voltage may comprise changing the voltage from a first voltage to a second (higher) voltage, and the nozzle may eject a droplet or spray of droplets when the voltage is the second (higher) voltage but not when the voltage is the first (lower) voltage.

The voltage may be a DC voltage or an AC voltage. The voltage may be a pulsed voltage. The variation of the AC voltage may be sinusoidal voltage or a square wave voltage, optionally with a constant frequency switch between V1 and V2. This has the effect of switching the spray on and off and slowing the volumetric throughput, which promotes the evaporation of any liquid in the droplets after deposition, minimising flow of the droplets after they have landed on the surface.

In some embodiments a backpressure may be applied to the liquid in the nozzle during nanoelectrospraying. This may be from a chamber in fluid connect from a nozzle that is pressurized. In an embodiment, the chamber may be a syringe or other pressurisable chamber in fluid connection with the nozzle. A backpressure of from 0.01 bar to 1 bar, optionally 0.01 bar to 0.1 bar may be applied. This may assist the ejection of the droplets. The pressure may be raised and lowered, e.g. in a cycle, during the nanoelectrospraying, optionally in synchronization with any cycle of the voltage variation.

The distance from the nozzle to the ocular prosthetic device during the nanoelectrospraying is preferably from 0.1 mm to 5 mm, preferably 0.5 mm to 4 mm, preferably 0.5 mm to 3 mm, preferably 0.5 mm to 2 mm, preferably about 1 mm. The smaller distance from the nozzle has been found to lead to a smaller surface area of the lens being coated with a spray of droplets. This leads to finer control of the pattern being produced on the ocular prosthetic device. It was found, for example, that a width of coating of less than 3 mm could be achieved with a nozzle to ocular prosthetic device distance of 3 mm, and that a width of coating of less than 1 mm could be achieved with a nozzle to ocular prosthetic device distance of 1 mm.

The distance from the nozzle to the position on the ocular prosthetic device at which a droplet (or the center of a spray of droplets) will be deposited (nozzle to substrate distance, NSD) during the nanoelectrospraying is preferably 5 mm or less, for example, 4.5 mm or less, 4 mm or less, 3.5 mm or less, 3 mm or less, 2.5 mm or less, 2 mm or less, 1.5 mm or less, 1 mm or less, 0.5 mm or less, or 0.1 mm or less. The distance from the nozzle to the ocular prosthetic device during the nanoelectrospraying is preferably from 0.1 mm to 4.5 mm, for example, 0.5 mm to 4 mm, 1 mm to 3.5 mm, 1 mm to 3 mm, 1.5 mm to 2.5 mm, 0.1 mm to 2 mm. In some examples, it was found that the width of coating increases linearly with the distance from the nozzle to the ocular prosthetic device and/or the thickness of the coating decreases linearly with the distance from the nozzle to the ocular prosthetic device. In some examples, the ocular prosthetic device is a contact lens and the NSD is the distance from the nozzle to a point on the surface of the contact lens where a droplet will be deposited (which may correspond to the center of the deposited active ingredient and/or carrier species on the surface of the contact lens).

The nozzle may have an inner diameter of 250 μm or less, optionally 200 μm or less, optionally 150 μm or less, optionally 100 μm or less. The nozzle may have an inner diameter of from 10 μm to 250 μm, optionally 10 μm to 200 μm, optionally 50 μm to 200 μm, optionally 50 μm to 150 μm, optionally 75 μm to 125 μm, optionally about 100 μm.

The flow rate of droplets from the nozzle may be from 0.1 nL/min to 5000 nL/min, optionally from 1 nL/min to 1000 nL/min, optionally from 1 nL/min to 100 nL/min.

During the nanoelectrospraying, the substrate (i.e., the ocular prothetic device) and the nozzle of the nanoelectrospraying equipment may move relative to each other. In some examples, the nozzle is stationary and the substrate is in motion. In some examples, the substrate is stationary and the nozzle is in motion. In some examples, both the nozzle and the substrate are in motion, for example, in opposite directions. The relative movement may be at a speed of at least about 0.1 mm/s, for example, at least about 0.5 mm/s, at least about 1 mm/s, at least about 1.5 mm/s, at least about 2 mm/s, at least about 2.5 mm/s, at least about 3 mm/s, at least about 3.5 mm/s, at least about 4 mm/s, at least about 4.5 mm/s, at least about 5 mm/s, at least about 5.5 mm/s, at least about 6 mm/s, at least about 6.5 mm/s, at least about 7 mm/s, at least about 7.5 mm/s, at least about 8 mm/s, at least about 8.5 mm/s, at least about 9 mm/s, at least about 9.5 mm/s, at least about 10 mm/s, at least about 15 mm/s, at least about 20 mm/s, at least about 25 mm/s, at least about 30 mm/s, at least about 35 mm/s, at least about 40 mm/s, at least about 45 mm/s, or at least about 50 mm/s. In some examples, the relative movement may be at a speed of up to about 50 mm/s, for example, up to about 45 mm/s, up to about 40 mm/s, up to about 35 mm/s, up to about 30 mm/s, up to about 25 mm/s, up to about 20 mm/s, up to about 15 mm/s, up to about 10 mm/s, up to about 9.5 mm/s, up to about 9 mm/s, up to about 8.5 mm/s, up to about 8 mm/s, up to about 7.5 mm/s, up to about 7 mm/s, up to about 6.5 mm/s, up to about 6 mm/s, up to about 5.5 mm/s, up to about 5 mm/s, up to about 4.5 mm/s, up to about 4 mm/s, up to about 3.5 mm/s, up to about 3 mm/s, up to about 2.5 mm/s, up to about 2 mm/s, up to about 1.5 mm/s, up to about 1 mm/s, up to about 0.5 mm/s, or up to about 0.1 mm/s. In some examples, the relative movement may be at a speed of from about 0.1 mm/s to about 50 mm/s, for example, about 0.5 mm/s to about 45 mm/s, about 1 mm/s to about 40 mm/s, about 1.5 mm/s to about 35 mm/s, about 2 mm/s to about 30 mm/s, about 2.5 mm/s to about 25 mm/s, about 3 mm/s to about 20 mm/s, about 3.5 mm/s to about 15 mm/s, about 4 mm/s to about 10 mm/s, about 4.5 mm/s to about 9.5 mm/s, about 5 mm/s to about 9 mm/s, about 5.5 mm/s to about 8.5 mm/s, about 6 mm/s to about 8 mm/s, about 6.5 mm/s to about 7.5 mm/s, or about 7 mm/s to about 50 mm/s. In some examples, it was found that the width of the coating was not significantly affected by the speed at which the substrate and/or the nozzle moved and/or that the thickness of the coating decreased (for a fixed number of layers, e.g., rotations of the nozzle or substrate) with the speed at which the substrate and/or the nozzle moved.

The droplets will typically include a liquid in which the active ingredient and/or the carrier species is dispersed, e.g. dissolved. A plurality of active ingredients and/or a plurality of carrier species may be present in the droplets, and then in the coating on the ocular prosthetic device. The liquid may be selected from a polar solvent and a non-polar solvent. The solvent may be a protic polar solvent or a non-polar protic solvent. The polar protic solvent may be selected from water, an alkanol, e.g. n-butanol, isopropanol, n-propanol, ethanol, and a carboxylic acid, such as formic acid or acetic acid. The polar non-protic solvent may be a solvent such a solvent selected from dichloromethane, tetrahydrofuran, ethylacetate, acetone, dimethylformamide, acetonitrile and dimethyl sulphoxide.

The ocular prosthetic device may have a concave surface and/or a convex surface and the droplets and the active ingredient and/or the carrier species may be applied to the concave surface and/or convex surface.

The carrier species may be a polymer, preferably a biocompatible polymer. The carrier species may be a polymer selected from sodium polystyrene sulfonate (PSS), polyethers, such as a polyethylene oxide (PEO), polyoxyethylene glycol or polyethylene glycol (PEG), polyethylene imine (PEI), a biodegradable polymer such as a polylactic acid, e.g poly-d,I-lactic acid, polycaprolactone, polyglycolic acid, poly(lactic-co-glycolic acid) (PLGA), zein, poly(I-lactide-co-ε-caprolactone) (PLCL), hyaluronic acid, polymethylsilsesquioxane (PMSQ) and copolymers, derivatives, and mixtures thereof. Other polymers that may be used include those well known to those of skill in the art to be used in cell cultures, implants, regenerative, therapeutic, and pharmaceutical compositions. One such example is polyvinylpyrrolidone (PVP).

The polymer may be a water-soluble and/or hydrophilic polymer, which may be selected from biocompatible polymers, including, but not limited to, cellulose ether polymers, including those selected from the group consisting of hydroxyl alkyl cellulose, including hydroxypropyl methyl cellulose (HPMC), hydroxypropyl cellulose (HPC), hydroxyethyl cellulose (HEC), methyl cellulose (MC), carboxymethyl cellulose (CMC), and mixtures thereof.

The polymer may also be selected from polyvinylpyrrolidone, vinyl acetate, polyvinylpyrrolidone-vinyl acetate copolymers, polyvinyl alcohol (PVA), acrylates and polyacrylic acid (PAA), including polyacrylate polymer, vinylcaprolactam/sodium acrylate polymers, methacrylates, poly(acryl amide-co-acrylic acid) (PAAm-co-AA), vinyl acetate and crotonic acid copolymers, polyacrylamide, polyethylene phosphonate, polybutene phosphonate, polystyrene, polyvinylphosphonates, polyalkylenes, and carboxy vinyl polymer. The multiphasic fiber compositions may comprise derivatives, copolymers, and further combinations of such polymers, as well.

The polymer may be selected from water insoluble or hydrophobic polymers including, but not limited to, cellulose acetate, cellulose nitrate, ethylene-vinyl acetate copolymers, vinyl acetate homopolymer, ethyl cellulose, butyl cellulose, isopropyl cellulose, shellac, hydrophobic silicone polymer (e.g., dimethylsilicone), polymethyl methacrylate (PMMA), cellulose acetate phthalate and natural or synthetic rubber; siloxanes, such as polydimethylsiloxane (PMDS), cellulose, polyethylene, polypropylene, polyesters, polyurethane and nylon, including copolymers, derivatives, and combinations thereof.

In a preferred embodiment, the carrier species is a polymer selected from PLGA, zein, PLCL, and hyaluronic acid.

Poly(lactide-co-caprolactone) (PLCL) is a co-polymer of poly(lactic acid) and polycaprolactone. The co-polymer is biocompatible and has biodegradable characteristics. They show high degradation and hydrophobicity as well as good miscibility with other polymers and the permeability is excellent for a drug-carrying polymer. These aliphatic polymers are approved by the FDA and as such are extensively used in the pharmaceutical sector.

Poly (Lactic-co-Glycolic Acid) (PLGA) 50:50, with acidic terminal group (used in the Examples). PLGA, approved by the FDA, is an extensively used polymer in the pharmaceutical industry as it has characteristics favourable to releasing drug in a controlled manner. The polymer is a synthetic biodegradable and non-immunogenic product that can have its physicochemical properties altered by changing the ratio of PGA and LA during its production. PGA is highly crystalline material and hydrophilic but when combined with LA becomes a polymer with a longer degradation time due to higher hydrophobicity and less swelling capacity.

Zein is a class of protein found in maize. The commercial source of zein is a fine yellow powder which partially soluble in water alone. It solubilizes in a mixture of ethanol (70-80% w/w) and water (30-20% w/w) due to it amphiphilic nature. It is a biodegradable and biocompatible material.

The carrier species, e.g. polymer, may be present in the droplets in a concentration of from 0.1 mg/ml to 100 mg/ml, optionally 1 mg/ml to 100 mg/ml, optionally from 5 mg/ml to 75 mg/ml, optionally from 10 mg/ml to 75 mg/ml, optionally from 10 mg/ml to 50 mg/ml, optionally from 50 mg/ml to 75 mg/ml.

The active ingredient may be or comprise a pharmaceutically active ingredient, optionally two or more pharmaceutically active ingredients. The pharmaceutically active ingredient may be selected from a small molecule drug, proteins, nucleic acids, polysaccharides, and biologics. A biologic may be selected from a vaccine, blood, blood components, cells, genes, tissues and recombinant proteins. A biologic may be selected from a protein that can control the action of other proteins and cellular processes, a gene that control the production of proteins, a hormone, which may be a modified hormone, and a cell that produces one or more substances that suppress or activate components of the immune system. A biologic may be referred to as a biologic response modifier. The pharmaceutically active ingredient may be any agent capable of providing a therapeutic or a prophylactic benefit to a human or animal wearing an ocular prosthetic device having the pharmaceutically active ingredient thereon or therein. In an embodiment, the pharmaceutically active ingredient is effective for treating diseases and disorders of the eye or an ocular disease or disorder. In non-limiting, exemplary embodiments, the pharmaceutically active ingredient is selected from an anti-infective agent (e.g., an antibiotic or antifungal agent), an anesthetic agent, an anti-VEGF agent, an anti-inflammatory agent, a biological agent (i.e. a biologic, as described above. The biological agent may be or comprise a macromolecular molecule, including, but not limited to, RNA, DNA, antibodies or fragments thereof, peptides, proteins and carbohydrates), an intraocular pressure reducing agent (i.e., a glaucoma drug), or a combination thereof. Non-limiting examples of drugs are provided below. The ocular disease or disorder may be selected from disorders such as post-surgical and post-trauma pain, glaucoma, uveitis, and hay fever allergy and dacrocystitus.

The active ingredient may be selected from growth factors, angiogenic agents, anti-inflammatory agents, anti-infective agents such as antibacterial agents, antiviral agents, antifungal agents, and agents that inhibit protozoan infections, antineoplastic agents, anaesthetics, anti-cancer compositions, autonomic agents, steroids (e.g., corticosteroids), non-steroidal anti-inflammatory drugs (NSAIDs), antihistamines, mast-cell stabilizers, immunosuppressive agents, antimitotic agents, or other drug. In some embodiments, the active agent may be a wetting agent, surfactant, ocular demulcent, electrolyte, buffer, or preservative. In some cases, an active agent may improve a wearer's comfort when wearing a ocular prosthetic device, e.g. contact lens.

The antibacterial agent may be selected from bacitracin, chloramphenicol, ciprofloxacin, erythromycin, moxifloxacin, gatifloxacin, gentamicin, levofloxacin, sulfacetamide, polymyxin B, vancomycin, tobramycin, or a combination thereof.

The antiviral agents may be selected from trifluridine, vidarabine, acyclovir, valacyclovir, famciclovir, foscarnet, ganciclovir, formivirsen, and cidofovir.

The antifungal agents may be selected from amphotericin B, natamycin, fluconazole, itraconazole, ketoconazole, and miconazole.

The antiprotozoal agents may be selected from polymyxin B, neomycin, clotrimazole, miconazole, ketoconazole, propamidine, polyhexamethylene biguanide, chlorhexidine, itraconazole.

The anesthetic agents may be selected from an aminoamide and an aminoester. The aminoamide may be selected from lidocaine, prilocalne, mepivacaine and ropivacaine.

The aminoesters may be selected from benzocaine, procaine, proparacaine, and tetracaine.

The autonomic agents may be selected from acetylcholine, carbachol, pilocarpine, physostigmine, echothiophate, atropine, scopolamine, homatropine, cyclopentolate, tropicamide, dipivefrin, epinephrine, phenylephrine, apraclonidine, brimonidine, cocaine, hydroxyamphetamine, naphazoline, tatrahydrozoline, dapiprazole, betaxolol, carteolol, levobunolol, metipranolol, and timolol.

The anti-inflammatory agents may be selected from a non-steroidal anti-inflammatory agent, and a steroidal anti-inflammatory agent. Non-limiting examples include glucocorticoids (e.g., dexamethasone, prednisolone, fluorometholone, loteprednol, medrysone, and rimexolone) and NSAIDS (e.g., diclofenac, flurbiprofen, ketorolac, bromfenac, and nepafenac).

The pharmaceutically active ingredient may be selected from ophthalmic antihistamines, ophthalmic prostaglandin analogs, ophthalmic α-adrenoreceptor agonists, ophthalmic p-adrenoreceptor agonists, ophthalmic carbonic anhydrase inhibitors and ophthalmic parasympathomimetic agents. The ophthalmic antihistamines may be selected from alcaftadine, azelastine, bepotastine, cetirizine, epinastine, ketotifen, and olopatadine. The ophthalmic prostaglandin analogs may be selected from latanoprost, travoprost, bimatoprost, tafluprost, and unoprostone isopropyl. The ophthalmic α-adrenoreceptor agonists may be selected from brimonidine and apraclonidine. The ophthalmic p-adrenoreceptor agonists may be selected from timolol, betaxolol, carteolol and levobunolol. The ophthalmic carbonic anhydrase inhibitors may be selected from acetazolamide, dorzolamide and brinzolamide. The ophthalmic parasympathomimetic agents may be selected from carbachol and pilocarpine.

In an embodiment, the active ingredient is selected from ketotifen fumarate, timolol and latanoprost and sodium hyaluronate.

Ketotifen fumarate has a molecular weight of 250.50, pKa 12.3 (acid), Log P 3.4. It is a second-generation antihistamine drug for treating seasonal allergic conjunctivitis (SAC). The physicochemical characteristics of KF suggest that this drug is slightly soluble in aqueous solutions (7.87 μg/ml) and as such will dissociate from a polymer layer and move into the aqueous environment via diffusion.

Timolol is a nonselective beta-adrenergic antagonist given in an eye drop solution to reduce intraocular pressure, or pressure in the eyes.

Latanoprost is a prostaglandin F2α analogue, is a selective prostanoid FP receptor agonist which reduces the intraocular pressure by increasing the outflow of aqueous humour.

Sodium Hyaluronate (HA) is a naturally occurring anionic non-sulfated glucosaaminoglycon linear polysaccharide which has been used as an artificial tear solution. Due to its hygroscopic nature, it provides treatment for ocular discomfort and dry eye syndrome. It has an important role in structural and biological roles in animal tissues. The long polysaccharide chain provides the viscoelasticity which enhances the tear stability.

The active ingredient may be a compound for increasing the comfort of the wearer and/or improving the hydration of the ocular prosthetic device, e.g. contact lens, and eye in contact with the ocular prosthetic device, such as hyaluronic acid.

The pharmaceutically active agent may be present in the droplets in a concentration of from 0.1 mg/ml to 50 mg/ml, optionally from 0.1 mg/ml to 20 mg/ml, optionally from 0.1 mg/ml to 10 mg/ml.

The amount of active ingredient deposited on the ocular prosthetic device may be an amount suitable for it to carry our its function, e.g. therapeutic or prophylactic function if the active is a pharmaceutical active ingredient. The active ingredient may, for example, be present on the ocular prosthetic device in an amount of from 0.1 μg to 100 μg, optionally from 1 μg to 50 μg.

The active ingredient may be a colouring agent for imparting a colour to the ocular prosthetic device, e.g. contact lens, for cosmetic purposes. The colouring agent may be selected from a solubilized leuco sulfate ester of 7,16-Dichloro-6,15-dihydro-5,9,14,18-anthrazinetetrone (C.I. Vat Blue 6), 16,23-Dihydrodinaphtho[2,3-a:2′,3′-i]naphth[2′, 3′:6:7]indole[2,3-c]carbazole-5,10,15,17,22,24-hexone (C.I. Vat Brown 1), N,N′-(9,10-Dihydro-9,10-dioxo-1,5-anthracenediyl)bisbenzamide (C.I. Vat Yellow 3), 16,17-Dimethoxydinaphtho[1,2,3-cd:3′,2′,1′-1n]perylene-5,10-dione (C.I. Vat Green 1), 6-Ethoxy-2-(6-ethoxy-3-oxobenzo[b]thien-2(3H)-ylidene)benzo[b]thiophen-3(2H)-one (C.I. Vat Orange 5).

The droplets may further include an inorganic salt to increase their conductivity. The conductivity of the droplet, measured in a sample of a bulk of the liquid of the droplets, may be from 0 to 1 μS/m, optionally from 0.1 μS to 10 mS/m, optionally from 0.1 μS to 100 mS/m, optionally from 0.1 μS to 10 mS/m, optionally from 0 to 200 μS/m, optionally from 5 μS to 100 μS/m, optionally from optionally from 10 μS to 30 μS/m. The inorganic salt may, for example, be an ammonium salt, such as a tetraalkylammonium halide, such as tetrabutylammonium bromide (TBAB) or an alkali metal salt, such as an alkali metal halide, such as sodium chloride.

The droplets may have a dynamic viscosity, measured at 20° C. and standard pressure, of from 1 cP to 10000 cP, optionally 1 cP to 1000 cP, optionally 20 cP to 1000 cP, optionally from 50 cP to 1000 cP, optionally from 100 to 1000 cP. The dynamic viscosity may be measured using any standard technique, e.g. using a capillary viscometer, a rotational viscometer (e.g. using ISO 3218.2) or a rolling ball viscometer.

The droplets, and hence the active ingredient and/or carrier species may be coated on the ocular prosthetic device in a predetermined pattern. The pattern may be an array of discrete dots, of any shape, lines or shaped blocks. Each dot may result from an individual droplet. The lines and shaped blocks may be formed from the coalescence of a plurality of droplets on the surface of the ocular prosthetic device. The array may itself form a shape, e.g. a line or shaped block, such as a ring. The dots or shaped blocks may be regular or irregular shapes, e.g. n-sided polygon, wherein n is 3 to 6, or circles, ovals, and rings. The pattern may also include one or more features selected from symbols, letters and numbers. In an embodiment, the ocular prosthetic device comprises, when viewed from above the ocular prosthetic device, a substantially circular or oval shaped body with an optic zone disposed centrally in the body, which sits on the cornea over the pupil once the ocular prosthetic device, e.g. contact lens, is fitted to an eye, and an outer zone, which sits over the cornea and sclera, The droplets, and the active ingredient and/or carrier species, may be coated on the ocular prosthetic device so that they leave a substantial portion, optionally all of, the optic zone, free of droplets, and the active ingredient and/or carrier species. The droplets, and the active ingredient and/or carrier species, may be coated on the ocular prosthetic device so that they form a ring around the periphery of the ocular prosthetic device, when viewed from above the ocular prosthetic device. The ring may be in the form of a continuous line or a discontinuous array of dots, of any shape, or lines. In an embodiment, at least 10%, optionally at least 20%, optionally at least 25% optionally at least 30%, optionally at least 35%, optionally at least 40%, optionally at least 45%, optionally at least 50% of the area of the ocular prosthetic device, on the side of the ocular prosthetic device coated, is free of droplets and active ingredient/carrier species, after the nanoelectrospraying of the ocular prosthetic device is complete, and this area is preferably in the central portion of the ocular prosthetic device (which would sit over the pupil of an eye, when in place on an eye).

After nanoelectrospraying the droplets comprising the active ingredient and/or the carrier species, any liquid from the droplets (e.g. the solvent in which the active ingredient and/or the carrier species were dispersed, e.g. dissolved) is preferably evaporated to leave just a layer comprising the active ingredient and/or the carrier species. If both a carrier species, e.g. a biocompatible polymer, and an active ingredient, e.g. a pharmaceutically active ingredient, are present on the ocular prosthetic device, this may allow a controlled release of the active ingredient to the eye once the ocular prosthetic device, e.g. contact lens, has been placed on the eye.

The thickness on the ocular prosthetic device of a layer comprising the active ingredient and/or the carrier species, after removal, e.g. evaporation, of any liquid from the droplets, is preferably 20 μm or less, preferably 15 μm or less, preferably 10 μm or less, preferably 5 μm or less. The thickness on the ocular prosthetic device of a layer comprising the active ingredient and/or the carrier species, after removal of any liquid from the droplets, is preferably 0.1 μm to 20 μm, preferably 0.5 μm to 15 μm, preferably 1 μm to 10 μm.

Optionally, multiple layers of droplets are nanoelectrosprayed onto the ocular prosthetic device. The thickness on the ocular prosthetic device of the active ingredient and/or the carrier species, after removal, e.g., evaporation, of any liquid from the droplets may vary linearly with the number of layers of droplets that are nanoelectrosprayed onto the ocular prosthetic device. In some examples, at least 10 layers are nanoelectrosprayed onto the ocular prosthetic device, for example, at least 15 layers, at least 20 layers, at least 25 layers, at least 30 layers, at least 35 layers or at least 40 layers are nanoelectrosprayed onto the ocular prosthetic device. In some examples, up to 40 layers to are nanoelectrosprayed onto the ocular prosthetic device, for example, up to 35 layers, up to 30 layers, up to 25 layers, up to 20 layers, up to 15 layers or up to 40 layers are nanoelectrosprayed onto the ocular prosthetic device. In some examples, from 10 to 40 layers are nanoelectrosprayed onto the ocular prosthetic device, for example, 15 to 35 layers, 20 to 30 layers, or 25 to 40 layers are nanoelectrosprayed onto the ocular prosthetic device. In some examples, each layer corresponds to a single rotation of the nozzle relative to the ocular prosthetic device (e.g., the contact lens) such that a layer of the active ingredient and/or the carrier species is formed on the ocular prosthetic device.

Optionally two or more active ingredients are coated onto the ocular prosthetic device. The active ingredients may perform different functions, e.g. having different therapeutic effects.

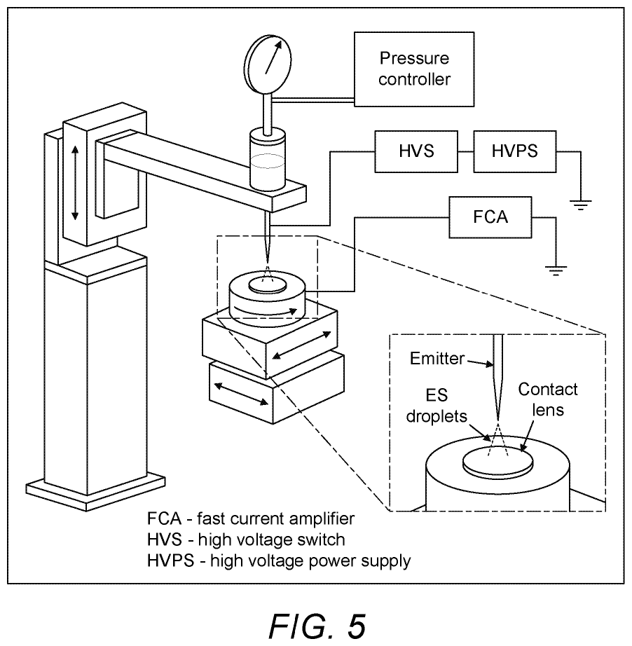

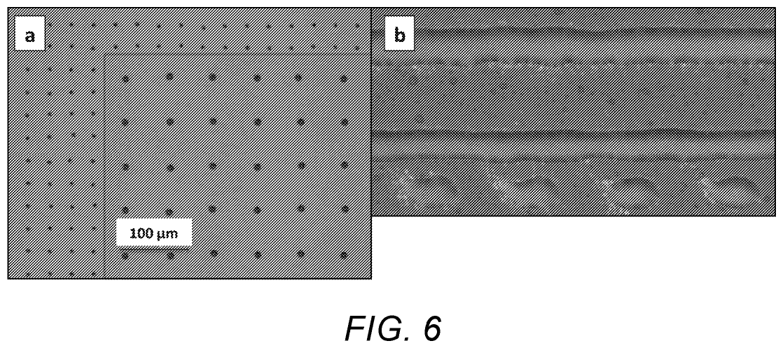

The pattern may be formed by moving the nozzle or moving the ocular prosthetic device, such that there is relative movement of the ocular prosthetic device with respect to the nozzle. The apparatus for carrying out the method may include a movable surface, on which the ocular prosthetic device resides during nanoelectrospraying. The movable surface may be movable in x and/or y directions (with the z direction being the direction from the surface to the nozzle), and x and y being perpendicular to one another and direction z. The movable surface may be movable in the x, y and z directions. Alternatively, or in addition, the nozzle may be movable in x and/or y directions (with the z direction being the direction from the surface to the nozzle), and x and y being perpendicular to one another and direction z. The nozzle may be movable in the x, y and z directions. In an embodiment, the surface on which the ocular prosthetic device reside is movable in x and y directions, while the nozzle is movable in the z direction. In an embodiment, the surface on which the ocular prosthetic device reside is movable in x and y directions, and in rotational translation (e.g. with the axis of rotation being through the centre of a circular or oval ocular prosthetic device), while the nozzle is movable in the z direction. This is depicted schematically in FIG. 5.

In an embodiment, the relative movement of the nozzle and the prosthetic device and/or the nanoelectrospraying (e.g. control of the voltage, which may cycle between V₂ and V₁ as described above) is controlled by a computer (which may be any electronic device for storing and processing data according to instructions given to it in a variable program). This can allow a desired pattern, determined digitally, to be translated to a pattern on a surface of the ocular prosthetic (or other prosthetic) device, and allow this to be formed on a plurality of devices in a consistent and reproducible manner, which may lend itself to mass production of the coated ocular prosthetic devices.

In a second aspect, there is provided an ocular prosthetic device formable according to the method of the first aspect. The ocular prosthetic device may be any device for placement on or in the eye or ocular region. The ocular prosthetic device may comprise a lens. The ocular prosthetic device may be selected from a contact lens, intraocular lens, a keratoprosthesis, intraocular ring, corneal inlay (also called an intracorneal implant), and an aqueous shunts/glaucoma filtration devices.

A contact lens may be considered to be a removable prosthetic lens for the eye. The contact lens may be to correct the vision of a wearer or for cosmetic reasons or for therapeutic reasons (other than vision correction). A contact lens is typically circular or oval in shape, when looked at from above one of its largest surfaces.

The intraocular lens may comprise an optic and at least one haptic. The intraocular lens may comprise two haptics. The optic is the portion of the intraocular lens which serves as the lens and the haptic(s) is(are) attached to the optic and maintains the optic in its proper place in the eye. The optic and the haptic(s) may be of the same or different material. A multi-piece lens is so called because the optic and the haptic(s) are made separately and then the haptic(s) is(are) attached to the optic. In a single piece lens, the optic and the haptic(s) are formed out of one piece of material. In this invention, a single piece lens is preferred.

The ocular prosthetic device, onto which the droplets or active ingredient and/or carrier species are coated, may comprise any suitable material. They may comprise a material selected from polymethylmethacrylate (PMMA), poly(4-methyl-pentene), styrene-acrylonitrile copolymer, acrylonitrile-butadiene-styrene copolymer, polycarbonate, polysulfone, cellulose acetate butyrate, silicone and fluorosilicone acrylate, and a hydrogel material. A hydrogel is a polymeric material that can absorb water while maintaining its structural integrity. The hydrogel material may comprise a polymer of 2-hydroxyethyl methacrylate (PHEMA), which may be a homopolymer of HEMA or co-polymer of HEMA and one or more other monomers, with the one or more other monomers optionally being selected from N-vinyl pyrrolidone, methyl methacrylate, methacrylic acid and phosphorylcholine, a polymer of vinyl alcohol (PVA), and a copolymer of methyl methacrylate and N-vinyl pyrrolidone. The hydrogel material may be a silicone hydrogel material. The hydrogel material, on which the droplets or active ingredient and/or carrier species are coated, may comprise water in an amount of from 10% to 80% by weight of the hydrogel material, optionally from 20% to 80% by weight of the hydrogel material, optionally from 20% to 60% by weight of the hydrogel material, optionally from 30% to 50% by weight of the hydrogel material, optionally from 35% to 45% by weight of the hydrogel material.

The contact lens may be a hard contact lens or a soft contact lens.

In a third aspect, there is provided a method of coating a surface of a prosthetic device, the method comprising

nanoelectrospraying of droplets comprising an active ingredient and/or a carrier species onto the surface in a predetermined pattern, the nanoelectrospraying involving controlling the flow rate of the droplets from a nozzle of the nanoelectrospraying equipment by controlling the voltage between the nozzle and the prosthetic device. The surface may be of any curvature, e.g. curved or flat. The surface may be electrically conducting, insulating or electrically semi-conducting. Preferably, the surface is a curved and/or electrically conductive surface.

The prosthetic device may be any prosthesis for a human or animal body, i.e. an object that will act as an artificial substitute or part for a human or animal body, which may be for functional or cosmetic reasons. The prosthetic device may be selected from a stent (i.e. a prothesis for a blood vessel), a heart valve, an artificial heart or any component thereof including power supply and regulator, a breast prosthesis, a larynx prosthesis, a trachea prosthesis, a tracheobronchial prosthesis, an ear or nose prosthesis, an internal organ prosthesis, biological tissue prosthesis, a limb, hand or foot prothesis. The manner of spraying may be as described for the ocular prosthetic device. All features of the process described for the ocular prosthetic devices are equally applicable to other prosthetic devices. The active ingredient and/or carrier species may be any suitable for the prosthesis. The active ingredient may be any pharmaceutically active ingredient for treating a disorder of the human or animal body, which may include any type of active ingredient mentioned herein.

In a fourth aspect, there is provided a prosthetic device formable according to the method of the third aspect. The surface may be of any curvature, e.g. curved or flat. The surface may be electrically conducting, insulating or electrically semi-conducting. Preferably, the surface is a curved and/or electrically conductive surface. The prosthetic device may be any device with a curved and/or electrically conductive surface, such as a stent. The electrically conductive surface may be any metallic and/or wet or water-containing surface, e.g. the surface of a hydrogel material (which may be as described above for the ocular prosthetic device). The prosthetic device may be a stent having a metallic surface, onto which the active and/or carrier species has been coated using the method of the third aspect. The pattern may be any suitable pattern, e.g. an array of dots or lines or block shapes.

### EXAMPLES

### Materials

Standard soft contact lenses, Biomedics 38 contact lenses (CooperVision Ltd, USA), with a composition of 62% polymacon/38% H₂O were used as the model lenses for coating. Ketotifen fumarate was supplied by Molekula, UK. Tetrabutylammonium bromide (TBAB), phosphate buffer saline solution tablets (pH 7.4) and poly(lactic-co-glycolic acid) 50:50 (PLGA) (brand name as Resomer RG502H) (MW7000-17000 Da) were obtained from Sigma-Aldrich, UK. Other polymers, poly(I-lactide-co-ε-caprolactone) 7:3 (PLCL, Ester terminated, Inherent viscosity: 0.93 dl/g) and poly-d,l-lactic acid (PDLLA, Ester terminated, Inherent viscosity: 0.66 dl/g) were supplied by Ashland, UK. Zein was obtained from Acros Organics, Thermo Fisher, UK), Sodium hyaluronate (MW 2 MDa) was obtained from Lifecore Biomedical. The solvents: Dichloromethane, Ethanol absolute, Acetone, Ethyl acetate were sourced from Sigma-Aldrich, UK. Milli-Q (Millipore, Merck, US) Ultra-pure water was used throughout all experiments. All materials were obtained from suppliers without further processing.

### Characterisation of the Solutions Used for nES

Conductivity measurements were performed by using Jenway 4510 conductivity meter with a micro volume conductivity probe. Interfacial tension of solution was measured by pendant drop method (Kyowa Interface Science, Model: DMs-401). The measurement runs for 100 second to collect the sufficient amount of data point for the analysis. For each sample, three measurements were carried out to calculate the average surface tension.

### Typical nES Process for Film Patterning

The nES process was monitored with a high-resolution camera with a high magnification zoom lens 6.5×(Thorlabs, UK). High voltage supply (F.u.G. Elektronik, Germany) was applied to the solution via a copper wire immersed in the solution. The voltage output and fast current amplifier were connected to a digital storage oscilloscope (Tektronix TBSD1000, RS component) to capture the voltage and current waveform respectively during a nES event. Different sizes of nozzles (50-100 μm) were connected to a 2 ml syringe, which was fixed on a manual 3D translation stage (as shown in FIG. 5). The ground substrate was fixed on a 2D motorised translation stage and its movement was controlled by a computer.

### Example 1: Nanoelectrospray Deposition of PLGA Polymer to Demonstrate Geometry Control of PLGA Polymer Film

### Methods

### PLGA Solution Preparation

A 1% w/v stock solution of PLGA was prepared by dissolving the polymer into acetone with stirring until a clear solution was formed. Different amount of tetrabutylammonium bromide (TBAB) was added into a glass vial containing 1 ml of the PLGA stock solution to produce solutions with a range of conductivity as listed in Table 1.

TABLE 1
PLGA solutions with different conductivity

| | 1% w/v PLGA in acetone | | |
|---|---|---|---|
| Solution | 1 | 2 | 3 |
| Conductivity (μS/m) | 1.09 | 19.98 | 113.8 |
| TBAB (mg) | 0 | <1 | 1 |
| Temperature (° C.) | 24.0 | 25.4 | 25.6 |

### Profilometry and Microscopic Analysis of nES Deposited Polymer Layer on Glass Substrate

Fluorine doped tin oxide glass slide was used for nES due to its conductive surface. After nES, profilometry (DektakXT Bruker) was used to measure the surface profile of the nES deposited polymer film. The Stylus radius 2 m and 1 mg Stylus force was used for the measurement. For microscopic images, Nikon LV150NL microscope with 5×-1000× magnification range was used to capture the sprayed polymer film on glass surface.

### Results

Nanoelectrospray (nES) Set Up and Operational Procedure

Nanoelectrospray printing was performed using the apparatus depicted in FIG. 5, where the fluid reservoir used was a disposable 2 mL syringe. The nES nozzle with tip diameter of 100 m was used. The nozzle to substrate distance (NSD) was varied from 1-5 mm and the resulting printed geometry of the deposited polymeric/drug film on the substrate was determined. At each NSD, an initial test was performed to determine the nES onset voltage, Von, by steadily increasing the applied voltage to the nozzle until nES initiation was detected visually (via the CCD camera imaging) and/or by detection of nES current from the fast current amplifier and oscilloscope. The lower nES-off voltage was selected to be between ground and Von and nES-on voltage, V2 was then set to a level above Vₒₙ in order to initiate nES deposition during the print cycle at a preselected frequency and duty cycle. In this example, a PLGA (1 wt % in acetone) with conductivity 19.98 μS/cm was used to nES coat a glass substrate with a FTO conductive surface layer. The voltage applied to the nozzle was controlled using the high-voltage switch to switch between V1 and V2 at a specific frequency and duty cycle, where the duty cycle typically ranged from 5 to 100% during printing.

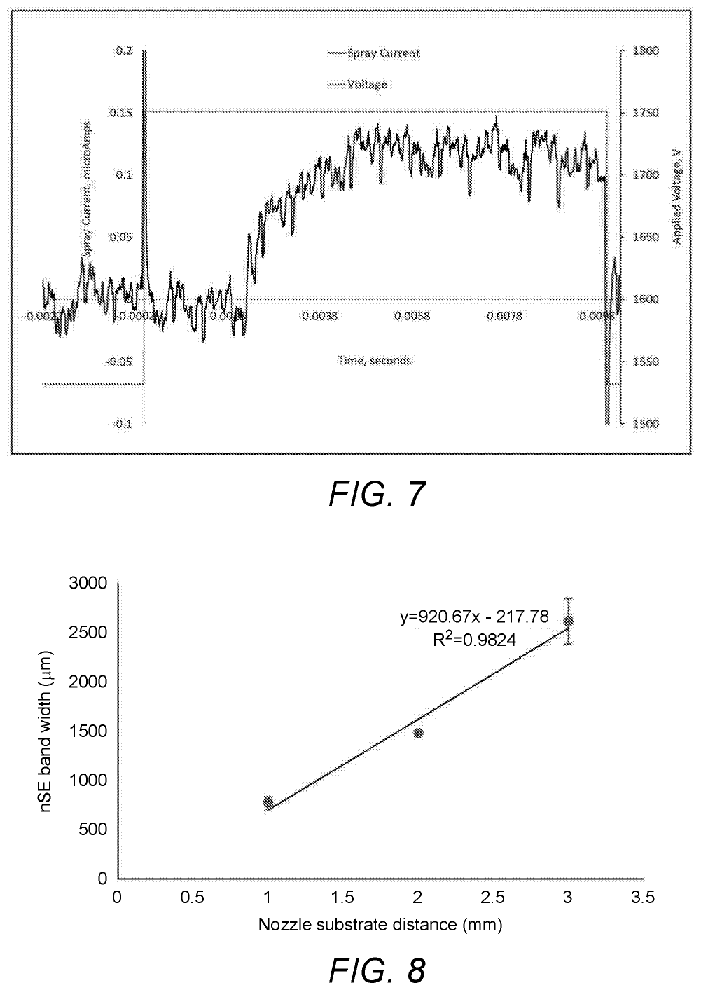

An example of the applied voltage waveform for the PLGA solution to be nES printed at 1 mm NSD is shown in FIG. 7, where V1 is 1532V, V2 is 1751V, the frequency is 10 Hz and the duty cycle is 10%. From the plot, the voltage is initially at a lower nES-off voltage, V1 of 1532V, where no fluid ejection from the nozzle is occurring. During this time the lens can be moved to a new print position without deposition occurring. At t=0, the voltage is raised to a nES-on voltage V2 of 1751V and after an initial nES formation time the nES current is seen to increase which corresponds to initiation of nES coating. At t=10 ms, the voltage is switch to the nES-off condition and the nES process is stopped. In order to produce a film coat of controlled geometry it is necessary to limit the delivery rate of polymer/drug solution to the substrate in order to prevent fluid build-up and achieve rapid drying of the film coat. If the delivery rate is too high, unwanted post-deposition flow over the surface of the substrate can occur (which can flow over the visual axis of the lens) or the fluid can dry to form a coffee-ring effect on the substrate. The delivery rate can be reduced in a number of ways during nES printing. For example, this can be achieved by increasing NSD, where under the same field strength and nES conditions the volumetric delivery rate of polymer containing fluid will be nES deposited over a larger surface area of the substrate. After nES at different NSD (1, 2, and 3 mm), polymer layers were dried overnight at room temperature before measuring the film geometry using surface profilometery. The results from nES printing at varying NSD are shown in Table 2 and FIG. 8 below.

As shown in FIG. 8, as the NSD is reduced the average width of the deposited polymer/drug film decreases. In this way the width of the film deposit can be controlled. As the throughput of polymer solutions is nominally the same at each condition, as the NSD is decreased the average thickness of the printed film increases, as shown in FIG. 8 and Table 2. By characterising the nozzle substrate distance and the spraying width with particular parameters, the spraying width can be predicted so that polymeric material is deposited on the area where it is not blocking the vision zoon of the contact lens.

TABLE 2
nES PLGA on glass substrate with different nozzle substrate distance.

| | PLGAₐ | PLGA_{b} | PLGA_{c} |
|---|---|---|---|
| NSD (mm) | 3 | 2 | 1 |
| Higher voltage (kV) | 2.122 | 1.999 | 1.796 |
| Lower voltage (kV) | 1.759 | 1.664 | 1.534 |
| Frequency (Hz) | 10 | 10 | 10 |
| Duty cycles (%) | 10 | 10 | 10 |
| Nozzle diameter (um) | 100 | 100 | 100 |
| Conductivity (uS/cm) | 19.98 | 19.98 | 19.98 |
| Platform speed (mm/s) | 0.2 | 0.2 | 0.2 |
| Width ± SD (μm)* | 2615.33 ± 233.09 | 1481.33 ± 20.79 | 774.00 ± 63.91 |
| Height ± SD (μm)* | 0.085 ± 0.009 | 0.122 ± 0.100 | 0.278 ± 0.024 |

*All profilometery data can be found in the FIGS. (see A5-A7)

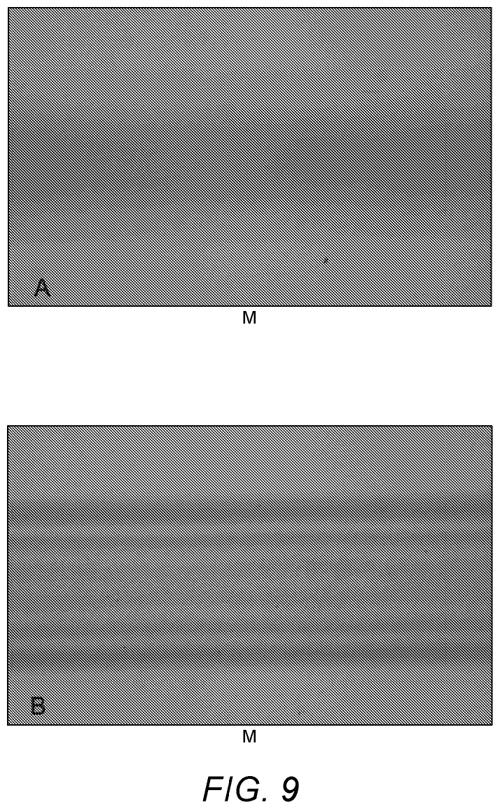

Once a suitable combination of nES process parameters of voltage, frequency, duty cycle, NSD and stage speed had been determined, the amount of deposition polymer/drug could be controlled and increased by successive overprinting onto the substrate. An example of the resulting film geometry from a single print pass and a five times overprinted substrate are detailed in FIG. 9.

From Table 3, the effect of successive overprinting is to increase the average thickness of the printed film. Although there appears to be a small increase in width, the printed features from single pass printing and five times overprinting appear to have near identical width from optical microscopy, as shown in FIGS. 9(A) and (B) below, and the discrepancy is likely due to difficulty in detecting the edge of the single-pass printed film due to it being only a few nm thick. The average thickness and peak height of nES printed layers after five overprints are found to be approximately 5 times higher than the single nES deposited layer of PLGA, as shown in Table 3 and the results shown in FIGS. 9(C) and (D) from profilometery.

TABLE 3
nES of 1% PLGA with different number of overprint cycles.

| | PLGA_{d} | PLGAₑ | PLGA_{f} | PLGA_{g} |
|---|---|---|---|---|
| Nozzle substrate | 1 | 1 | 1 | 1 |
| distance (mm) | | | | |
| Higher voltage (kV) | 1.796 | 1.796 | 1.751 | 1.751 |
| Lower voltage (kV) | 1.534 | 1.534 | 1.532 | 1.532 |
| Frequency (Hz) | 10 | 10 | 10 | 10 |
| Duty cycles (%) | 10 | 10 | 10 | 10 |
| Nozzle diameter (um) | 100 | 100 | 100 | 100 |
| Conductivity (uS) | 19.98 | 19.98 | 19.98 | 19.98 |
| Platform speed (mm/s) | 1 | 1 | 1 | 1 |
| Over print (times) | 1 | 5 | 1 | 5 |
| Width ± SD (μm)* | 685.00 ± 45.21 | 778.3 ± 28.29 | 557.67 ± 57.98 | 695.33 ± 19.66 |
| Height ± SD (μm)* | 0.030 ± 0.003 | 0.278 ± 0.054 | 0.043 ± 0.013 | 0.244 ± 0.015 |

*All profilometery and microscopic data can be found in the FIGS. (see A8-A10 and A12-A15)

Alternatives controlling nES fluid delivery rate to the substrate to achieve rapid drying are by decreasing the duty cycle and/or print frequency, increasing the stage speed or by reducing the nES-on voltage V2.

### Example 2: Nanoelectrospray Deposition of Drug-Polymer Films onto Contact Lenses

Method

### Drug-Polymer Solution Formulation Preparation

Table 4 depicts the nES formulation of different polymers with or without ketotifen in organic solvents. While sodium hyaluronate (HA) was dissolved in water/ethanol (1/1) mixture and nES deposited without any drug molecule to demonstrate the spraying capabilities of high molecular weight polymer via nES technique.

TABLE 4
Example drug-polymer solutions used to nES onto contact lenses

| | Polymer | Ketotifen | | | |
|---|---|---|---|---|---|
| | concentration | concentration | EA | DCM | Ethanol |
| Sample name | (mg/ml) | (mg/ml) | (ml) | (ml) | (ml) |
| PLCL 70:30 | 28.23 | 2.88 | 0.4 | 0.15 | 0.5 |
| PDLLA | 24.33 | 2.40 | 0.5 | 0.25 | 0.5 |

| | | | |
|---|---|---|---|
| Zein 1 | 25.24 | 2.62 | 1 ml of ethanol/water |
| | | | (8/2 w/w) |
| Zein 2 | 50.03 | 5.07 | 1 ml of ethanol/water |
| | | | (8/2 w/w) |
| HA | 1.00 | 0.00 | 1 ml water/ethanol |
| | | | (0.5/0.5 mL) |

nES of Drug-Polymer Solutions onto Contact Lenses

Commercial contact lenses were removed from the packaging and soaked in Milli-Q water to remove saline solution on the lens. Excess water on the lens was removed by lint-free dry wipe before being transferred onto a conductive glass substrate for nES. The drug-polymer solution was sprayed on the contact lens via nES with the parameters as shown in Table 5. A set of two lens was prepared for in vivo drug release experiment and another set was used to quantify the amount of drug being deposited on contact lens via nES.

TABLE 5
Parameters of nES on contact lenses.

| | PLCL 7:3 | PDLLA | Zein 1 | Zein 2 | HA |
|---|---|---|---|---|---|
| Nozzle substrate | 3.2 | 3.5 | 3.5 | 3.5 | 3.5 |
| distance/mm | | | | | |
| Voltage/kV | 2.289 | 1.724 | 2.277 | 2.520 | 2.029 |
| Nozzle inner | 50 | 100 | 100 | 100 | 100 |
| diameter/μm | | | | | |
| Pressure/bar | 0.094 | 0 | 0 | 0 | 0.037 |
| Revolution/turns | 30 | 20 | 20 | 20 | 20 |
| Dose speed | 15 | 20 | 20 | 20 | 5 |
| Dose radius/mm | 4.5 | 4.5 | 4.5 | 4.5 | 4.5 |

The total amount of ketotifen deposited on a contact lens was determined by immersing the processed lens into 1 ml of the original solvent used for solubilising both the polymer and ketotifen. 0.2 ml of the solution was measured using UV absorbance at 300 nm by a quartz microplate to quantify the drug loading on the lens after nES.

### In-Vitro Drug Release

Glass vials with screw caps containing 2 ml PBS pH 7.3 were first placed in a shaking incubator (IKA KS 2000i Control) set at 125 rpm and 35° C. for 2 hours to equilibrium to the environmental temperature. The contact lens was transferred immediately to the vial after the nES process for drug release study. 0.5 ml of the release sample was pipetted and replaced by same amount of PBS. At pre-determined time intervals, 0.2 ml PBS was taken and placed into a quartz microplate (Hellma, Germany). Ketotifen release was measured using UV absorbance at 300 nm using a microplate reader and analysed using the MARS Data Analysis software (ClarioStar multiwell plate reader, BMG-Labtech, UK).

### Optical Clarity of nES Coated Contact Lenses

The transmission of light through contact lenses was studied using a lightbox (Schott, KL1500 Electronic, MA USA), charged coupled device (CCD) camera (UVP, Cambridge, UK) and the Genesnap image capture program (Syngene, Cambridge, UK). Contact lenses were coated as described and placed onto PBS soaked gauze to maintain hydration and enclosed in a petri dish. Lenses were placed onto a petri dish base and centred over the light source. Images of control lenses (no deposition) and nES processed lenses were captured.

### Morphological Studies by Scanning Electron Microscopy (SEM)

Images of the surface and cross section of nES lenses were taken using SEM (Zeiss, Gemini300, UK). Immediately after nES deposition, lenses were cut in half. Lenses were allowed to dehydrate and the dried lenses placed onto SEM stubs. Samples were prepared by gold sputter coating.

### Results

### nES Deposition Mass Measurement

In order to assess the quality of the materials that is deposited on the contact lens by nES, each solution was first sprayed on a contact lens, then dissolved into 1 ml of the solvent that can completely solubilise both the model polymer and model drug on the lens. The amount of the model drug (ketotifen) deposited on the contact lens was quantified by measuring the absorbance of 200 μl of the sample at 300 nm using a quartz microplate. The average value of two lens were calculated with standard deviation (SD). As it can be seen in Table 6, the SD is very low for the model drug loaded in different polymers. This indicates that the nES material deposition is highly accurate and reproducible.

### Characterisation of nES Coated Contact Lenses

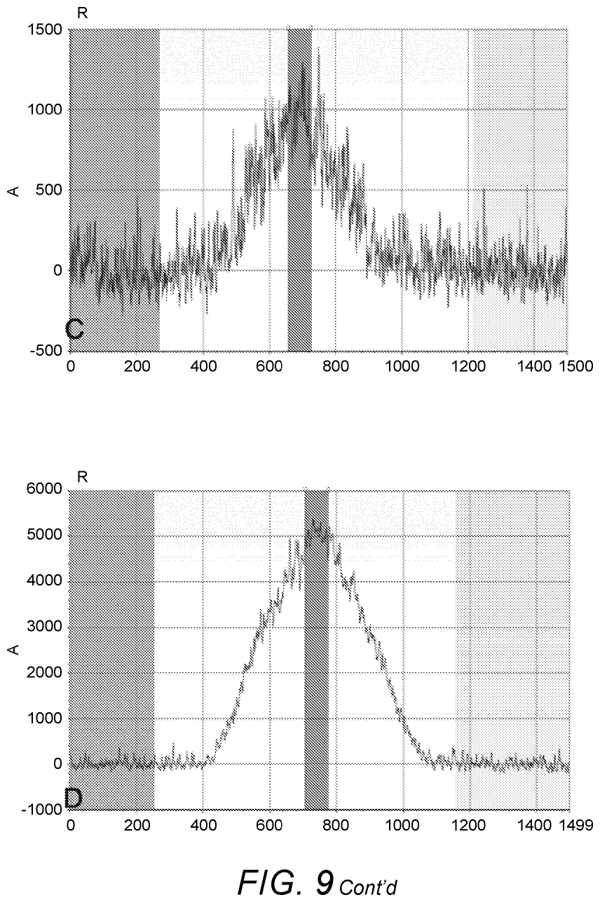

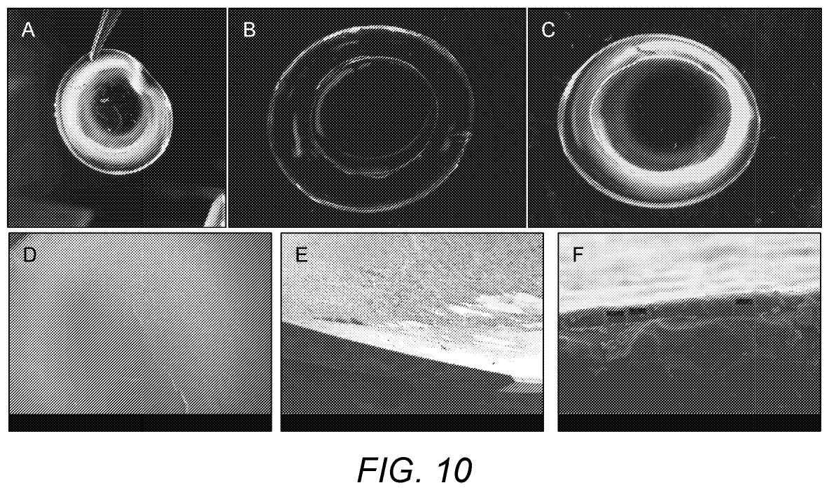

Using ketotifin fumarate as the model drug and PLCL as the model polymer, the accuracy of the nES liquid deposition around the periphery of the lens and its physical effect on the optical clarity were investigated. Using the nES parameters detailed in Table 5 to deposit a layer of the formulation containing PLCL7:3 with 10% Ketotifen Fumarate loading (PLCL/KF) creates a continuous white band after 30 rotations (FIG. 10a). Contact lenses are by their very nature, transparent and allow the transmission of light unhindered. FIG. 10b shows the excellent optical clarity of a freshly opened non-coated contact lens. On this CCD camera image, opacities are viewed as white areas. The contact lens that was nES with polymer and drug (FIG. 10c) demonstrates clear regions in the centre of the vision zone and a white band where the nES deposited the material. The central area of the contact lens is clear when viewed using this technique suggesting that vision would be unhindered.

Further detailed work using SEM further demonstrates the contrast between the smooth surface of the contact lens and the layer of polymer and deposition (FIGS. 10d and e). Through SEM observations, the deposited layer depicts a rough topology which is not seen by eye or using the CCD camera. Magnification of ×2000 at the cross-section (FIG. 10f) shows a typical region of deposition and its thickness is approximately 4 μm. It is essential that the thickness of the deposited band is not being too thick to cause irritation to the surface of the eye. The nES shows promise in that it is depositing such a thin peripheral layer, even after 30 rotations.

### In Vitro Drug Release

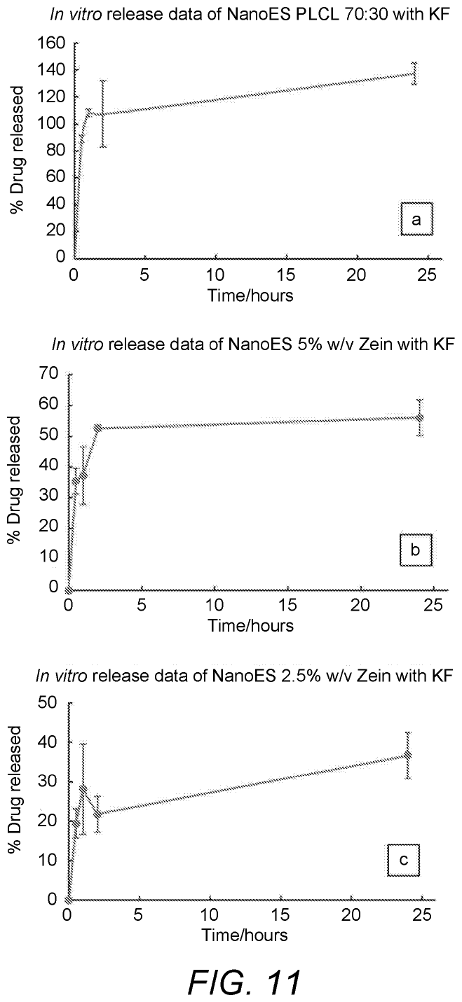

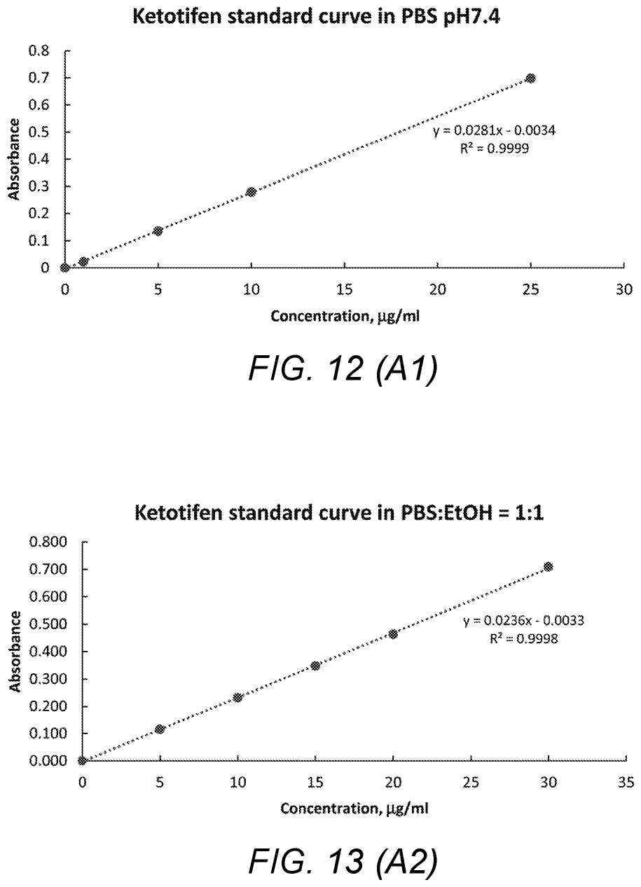

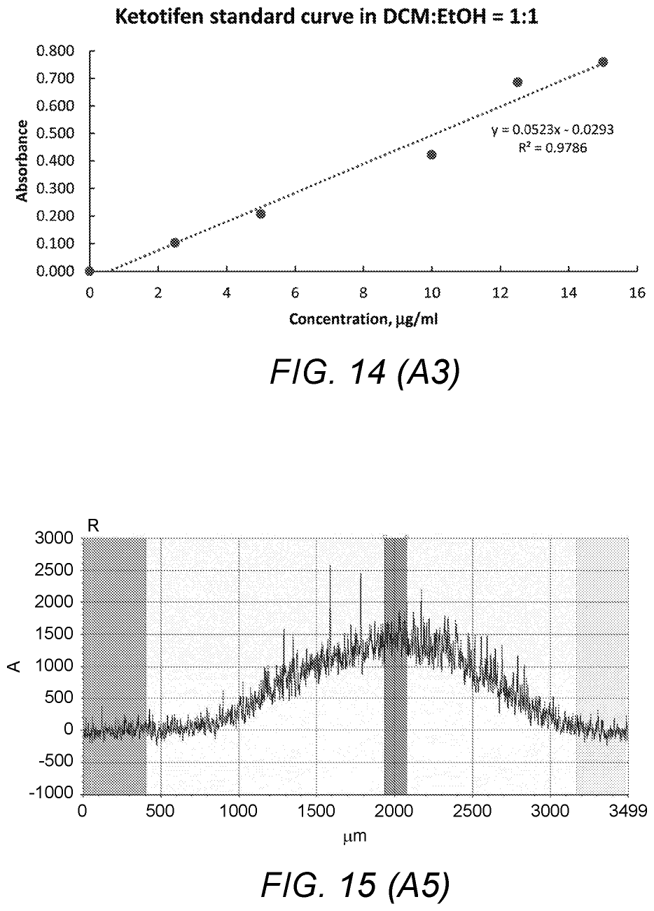

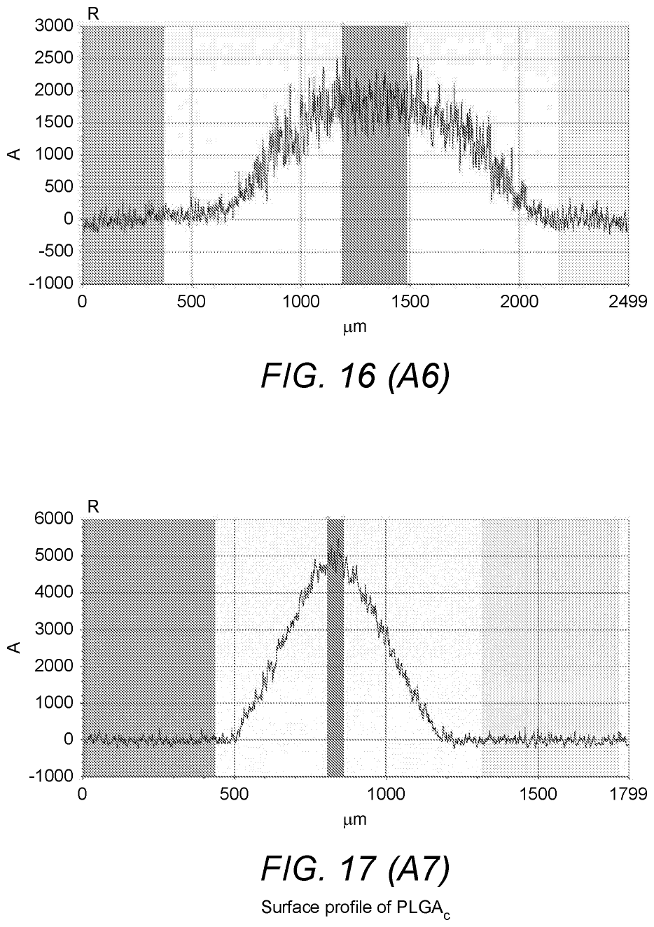

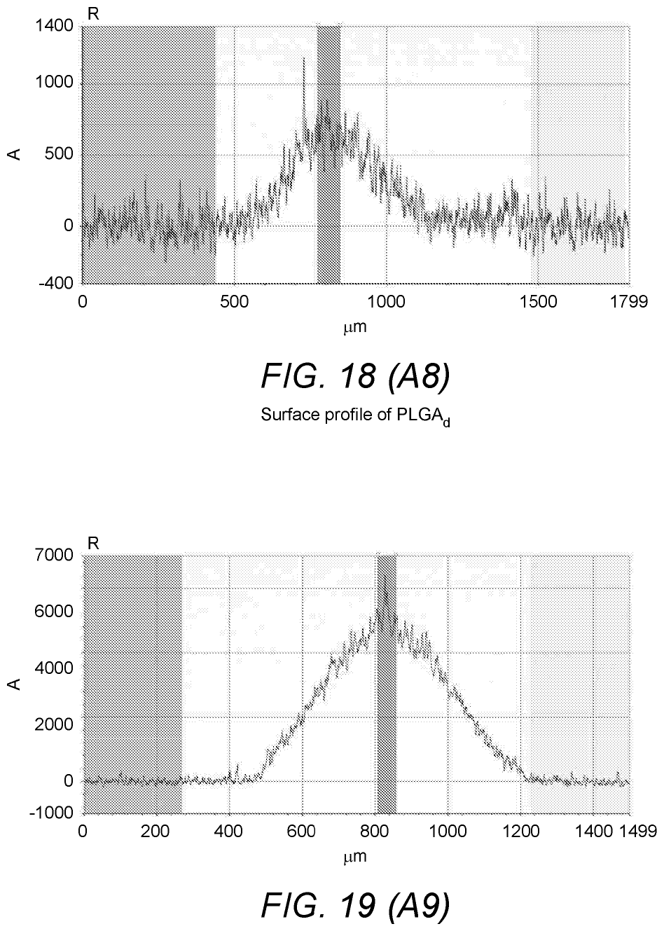

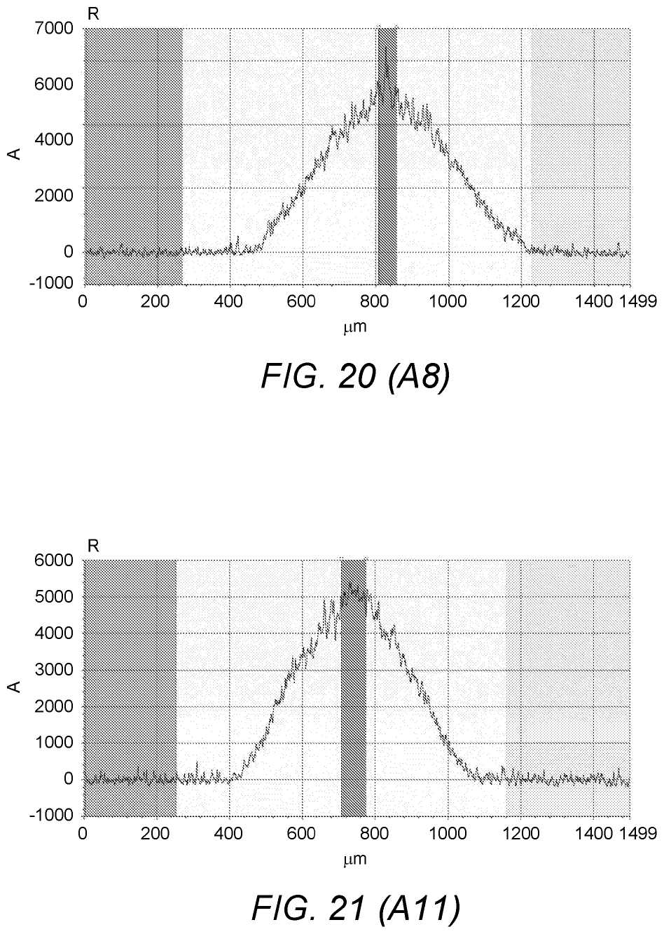

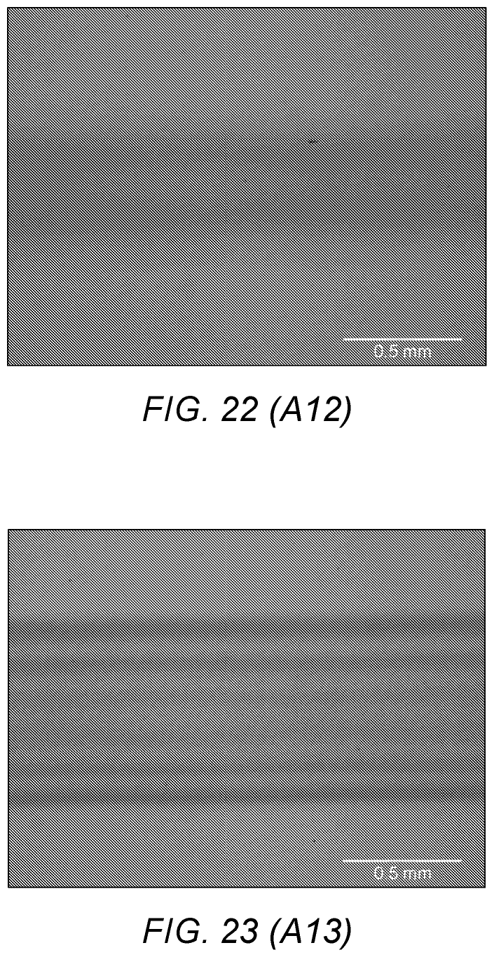

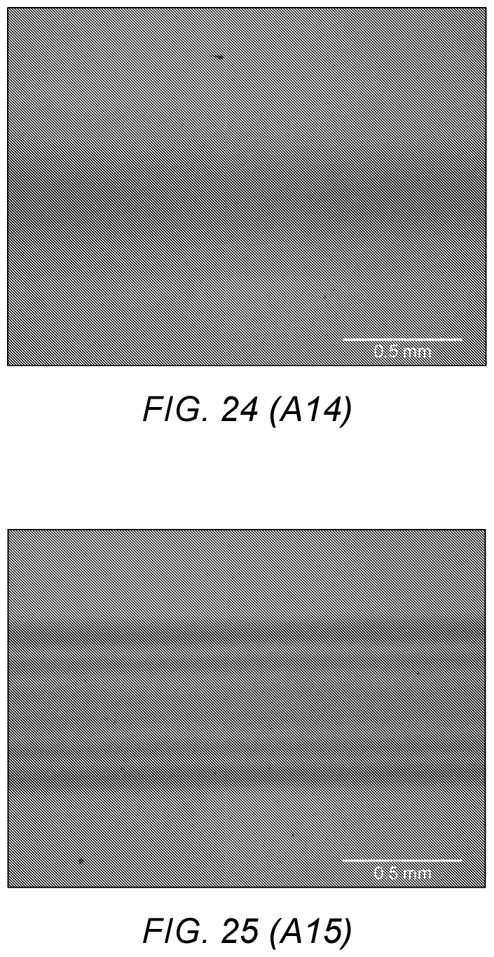

The in vitro drug-release profile of model drug (ketotifen) with model polymer deposited on the contact lenses are shown in FIG. 11. The % drug release of all release profile was calculated by the amount of ketotifen released at particular time interval divided by the experimentally determined value stated in Table 6. For PLCL 70:30, 25 μg of ketotifen was deposited on the lens after nES 30 revolutions. The release has achieved 80% in the first 30 minutes and reached 100% release in 1 hour. For zein solution, there is low level of the burst release in the first 30 minutes, followed by a slow release up to 24 hours.

TABLE 6
Quantity of ketotifen nES deposited on contact lens.

| | Average amount of | Standard deviation |
|---|---|---|
| Solution | ketotifen (μg) | (μg) |
| PLCL 70:30 | 25.00 | 0.18 |
| PDLLA | 2.34 | 0.39 |
| Zein | 8.13 | 0.45 |

Unless otherwise indicated, the properties mentioned herein are measured at room temperature and pressure.

### CONCLUSION

In this study, nES was developed to allow precise and reproducible liquid deposition of materials on to the conductive surfaces. Using the wet contact lens as the example coating system, our data demonstrated that the nES method developed can reproducibility deposit materials in a range of patterns as dots or lines or rings. The quantity of the polymer and drug deposited on the contact lens is highly reproducible. The deposited materials allow a range of release patter of the drug to be released from the coated lenses.

### Example 3—Further Validation of the Nanoelectrospray (nES) Process

### Materials

Zein was obtained from Acros Organics, Thermo Fisher, UK. Ethanol absolute was sourced from VWR Chemicals, UK. Milli-Q (Millipore, Merck, US) ultra-pure water was used throughout all experiments. Phosphate buffer saline tablet and methyl blue were purchased from Sigma-Aldrich, UK. All materials were obtained from suppliers without further processing.

### Methods

Preparation of Zein Solutions for Nanoelectrospraying (nES)

Zein solution was used as the example polymer solution to validate the nES process. As shown in Table 7, 2.5% w/v and 5% w/v zein solutions were prepared by dissolving zein powder in different % w/w aqueous ethanol with stirring at ambient condition until all powder dissolved. The solution was filtered through a 0.4 μm syringe filter (Fisher, UK) before nES.

TABLE 7
Composition and physicochemical properties of zein solutions.

| | Zein A | Zein B | Zein C | Zein D |
|---|---|---|---|---|
| Zein (% w/v) | 2.5 | 2.5 | 5 | 5 |
| Ethanol (% w/w) | 70 | 80 | 70 | 80 |
| Conductivity (μS/cm) | 200.0 | 186.2 | 333.0 | 293.0 |
| Density ± SD (g/ml) | 0.874 ± 0.001 | 0.848 ± 0.001 | 0.880 ± 0.001 | 0.857 ± 0.001 |
| Surface tension ± SD | 25.7 ± 1.0 | 24.9 ± 0.8 | 25.5 ± 1.2 | 24.7 ± 1.0 |
| (mN/m) | | | | |
| Viscosity ± SD (mPa · s) | 4.20 ± 0.05 | 3.87 ± 0.06 | 5.60 ± 0.09 | 5.15 ± 0.05 |

### Physiochemical Properties of Zein Solutions

Conductivity: Conductivity measurements were performed by using Jenway 4510 conductivity meter with a micro volume conductivity probe.

Viscosity: Dynamic viscosity of the solutions was measured by a Discovery HR-2 (TA instrument, Delaware, USA) equipped with a 2°, 40 mm cone-and-plate geometry. The method was set to be a flow ramp procedure from 0.1 to 100 s⁻¹ at 25° C. for 60 seconds.

The measurement was done in triplicate to calculate the average viscosity±SD at 80 s⁻¹.

Surface tension: Surface tension of the solutions was measured by pendant drop method (DMS-401, Kyowa interfacial science, Japan). For each sample, ten measurements were carried out to calculate the average surface tension±SD.

Density: Density of the solutions was measured by a density meter (DMA 4500M, Anton Paar, Germany) equipped with an oscillating U-tube. The measurement was done by injecting 1 ml of the sample to the system at 25° C. The measurement was done in triplicate to obtain the average density±SD.

### Experimental Setup of Nanoelectrospray

A device constructed as shown in FIG. 5 was used that consists of a nozzle, a high voltage supply (F.u.G. Elektronik, Germany), a digital camera with high magnification lens (Thorlabs, UK) and a ground substrate. A current amplifier (DLPCA—200, Laser Components, UK) and a digital storage oscilloscope (TBS1104, Tektronix, USA) were included in the experimental set up to monitor the current of the spraying process. Direct current was adopted in all experiment to simplify and understand the influence of spraying parameters on the quality of polymer film. A ceramic nozzle with a 50 μm internal diameter (MicroDot tip, 7364054, Nordson EFD, UK) was connected to a 2.5 ml luer-lock syringe (Terumo, Japan), which was fixed to a translation stage moving in the z-axis (the vertical axis) only. The fluorine doped tin oxide glass slide (Sigma-Aldrich, UK) was secured on a 2-dimension motorised translation stage. All the x, y, z movement and spraying parameters were controlled from the built-in control panel of the machine.

### Adjustable Spraying Parameters of Nanoelectrospraying Process

There are a few spraying parameters available to be adjusted from the system, which require clarification. The motion of the 2-dimensional motorised translation stage was controlled so the substrate moved relative to the nozzle along a circular path, allowing the radius, substrate speed and number of rotations to be defined as follows. The radius indicates the relative distance between the centre of a circle at pre-set x, y coordinates to its perimeter (which corresponds to the location of the nozzle), which enables deposition of polymer film with different size rings or dots. The number of rotations controls the number of layers of spray being deposited on the surface, which enables building up of the film thickness. The substrate speed indicates the speed of the movement of the substrate in a circular motion, with a higher number indicating a faster moving speed. The nozzle substrate distance indicates the distance between the apex of the nozzle and the substrate, which is controlled by changing the coordinate of the translational stage moving in the z-axis.

A range of combinations of spraying parameters were investigated in order to understand the influence of each parameter on the width and thickness of a deposited polymer film.

These parameters are:
1. Nozzle substrate distance (NSD) (1.5-4.5 mm)
2. Substrate speed (10-40 mm/sec)
3. Number of rotations (10-40)

### Surface Profile Measurement of Nanoelectrosprayed Zein Film Using a Contact Profilometer

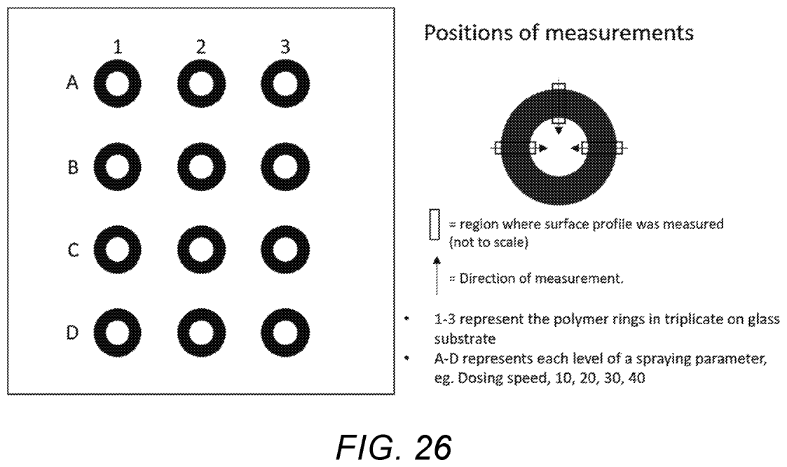

After nanoelectrospraying zein solutions on the conductive glass substrate, a stylus profilometer (DektakXT, Bruker, MA, USA) was used to measure the surface profile of the polymer films using the ‘Hills and Valleys’ profile. A stylus with 2 m radius was used for the measurement set at 1 mg stylus force. The measurement of spraying width and average step height (thickness) of the polymer film was performed at 3 locations as shown in FIG. 26, with a total number of 9 measurements for each experimental setting.

### Statistical Analysis

Statistical analysis was carried out using SPSS computer programme (SPSS Statistic 25). The difference in spraying width of two polymer concentrations and the viscosity difference were analysed by independent t-test (2 tailed) where a difference is considered when P<0.05 (95% confidence).

### Results and Discussion

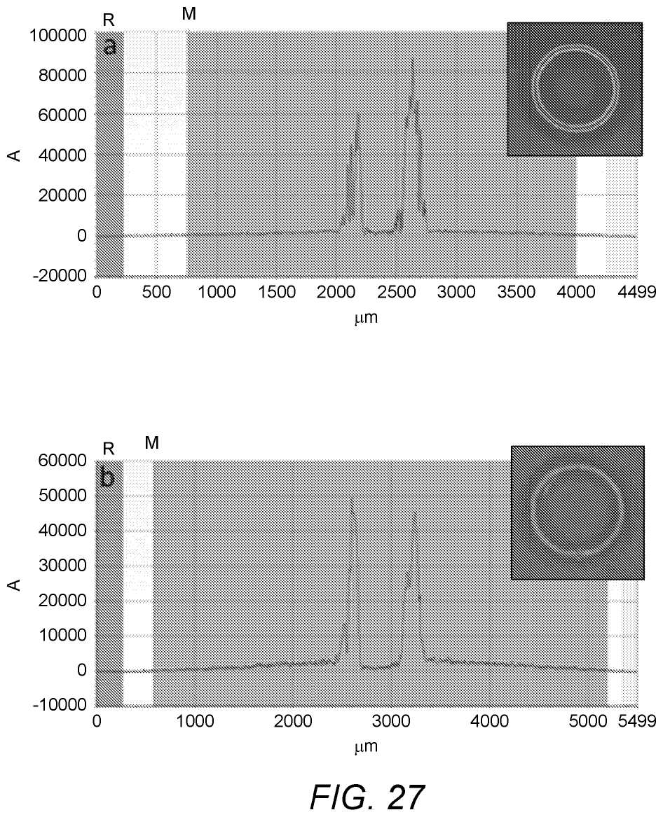

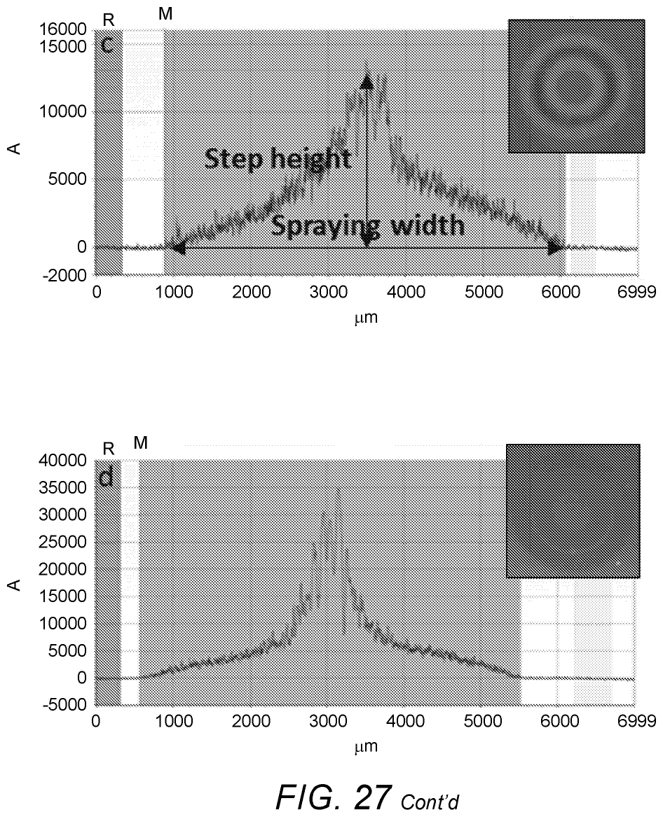

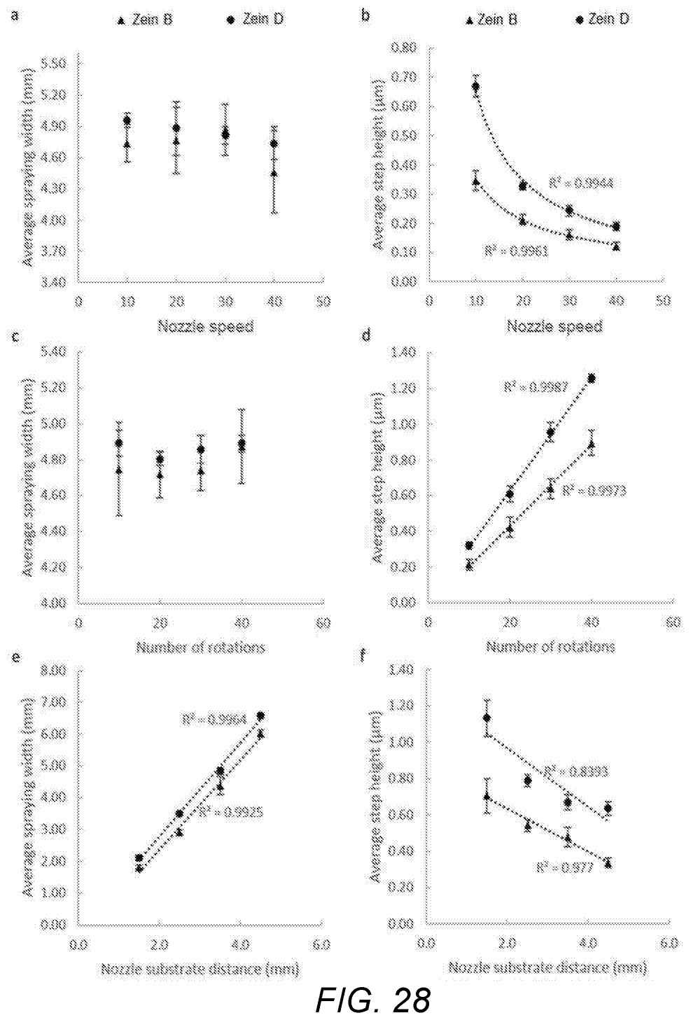

The zein solutions with two different concentrations were nanoelectrosprayed onto a conductive glass substrate to investigate the influence of polymer concentration on the morphology of the film formed. The zein solutions were sprayed with the same spraying parameters with different applied voltage (see Table 8). Profilometry results from FIG. 27 showed that the zein A and B solution (2.5% w/v in 70% and 80% w/w aqueous ethanol, respectively) formed a thin film on a glass substrate with two sharp peaks close to the centre, implying formation of a dried polymer layer with 2 raised edges. This formation can be explained by the coffee-ring effect occurring during the drying stage and can be minimised or removed by adjusting the drying speed of the nanoelectrosprayed fluid. The surface profile of both zein C and D (5% w/v, 70% and 80% w/w aqueous ethanol, respectively) showed a gradual increase of thickness from the edge of the film to the centre. The distance between the apex of the nozzle and the centre of the film is the shortest distance for nanoelectrosprayed droplets to travel and thus the thickness of the film is expected to be higher at the centre.

TABLE 8
Nanoelectrospraying parameters of
zein solutions showed in FIG. 27

| | Zein A | Zein B | Zein C | Zein D |
|---|---|---|---|---|
| Nozzle substrate | 3.5 | 3.5 | 3.5 | 3.5 |
| distance (mm) | | | | |
| Substrate speed | 10 | 10 | 10 | 10 |
| Number of revolutions | 10 | 10 | 10 | 10 |
| Voltage (kV) | 2.357 | 2.561 | 2.545 | 2.566 |

Different nanoelectrospraying parameters were investigated to understand their influence on the spraying width (the width of the ring-shaped film layer deposited on the substrate (a contact lens), illustrated in FIG. 27C and the step height (the thickness of the polymer film). When the film layer shows the coffee-ring effect, the spraying width is the total width of the coffee-ring shaped film layer and the step height is the average of the two thicknesses. Two concentrations of zein solution were nanoelectrosprayed with different spraying parameters to compare the influence of the polymer concentration on the film thickness and spraying width. FIG. 28 shows the average spraying width and step height of films formed from zein B and D solutions with different substrate speeds, number of rotations (i.e., layers) and NSD. As expected, the spraying width does not change with the substrate speed in the range of from 20-40 mm/s when the NSD remains constant, as shown in Table 9. The average step height of both zein D and zein B complied with the power law with a good correlation coefficient, indicating that the thickness of the film can be controlled by adjusting the substrate speed and solution concentration.

TABLE 9
Results of independent t-tests comparing
the mean spraying width of zein B and D

| Substrate speed (mm/s) | 10 | 20 | 30 | 40 |
|---|---|---|---|---|
| P value | 0.007 | 0.406 | 0.773 | 0.078 |
| Number of rotations | 10 | 20 | 30 | 40 |
| P value | 0.141 | 0.091 | 0.023 | 0.801 |
| NSD (mm) | 1.5 | 2.5 | 3.5 | 4.5 |
| P value | 0.0001 | 0.0001 | 0.0001 | 0.0001 |

For a particular formulation, the number of rotations is the main parameter that controls the thickness of the polymer film. By increasing the number of rotations, a layer by layer deposition of nanoelectrosprayed particles stack on each other to build up the thickness of polymer film. The step height (thickness) of zein film produced by the zein solution with both concentrations shows a linear relationship with the number of rotations. The results indicate that the thickness of polymer film can be controlled by the number of rotations.

For a particular formulation, the NSD is the main spraying parameter that determines the spraying width. It can be seen from FIG. 28 that the spraying width increases linearly with NSD for both polymer concentrations. The achieved spraying width for Zeins A to D with the tested NSDs ranged from 0.3-0.5 mm. Given that the voltage was adjusted at each NSD to achieve the same field strength to initiate nanoelectrospraying, the increase in NSD provides increased coverage of polymer on the substrate. The thickness of the film reduced with increasing NSD of from 1.5 to 4.5 mm. These results confirm that the film thickness can also be adjusted by adjusting the NSD.

### Example 4—Further Examples of Using Nanoelectrospray to Deposit Drug-Polymer Formulations onto Contact Lenses

### Materials

Commercial soft contact lenses, Biomedics 55 contact lenses (CooperVision Ltd, USA), with a composition of 45% ocufilcon D/55% H₂O were used as the model lenses nanoelectrospraying. Ketotifen fumarate and bimatoprost were purchased from Molekula (UK). Phosphate buffer saline (PBS) solution tablets (pH 7.4), triethylamine (≥99.5%), phosphoric acid (≥85%) were obtained from Sigma-Aldrich (UK). Zein was sourced from Acros Organics, Thermo Fisher (UK). Ethanol absolute was obtained from VWR (UK). Milli-Q (Millipore, Merck, US) ultra-pure water was used to prepare all aqueous solutions. Methanol and acetonitrile, high-performance liquid chromatography grade, were purchased from Fisher Scientific (UK). All materials were obtained from suppliers without further processing. 50 μm ceramic MicroDot tip (7364054) was purchased from Nordson RFD (UK).

Nanoelectrospraying (nES) of Drug-Polymer Solutions onto Contact Lenses

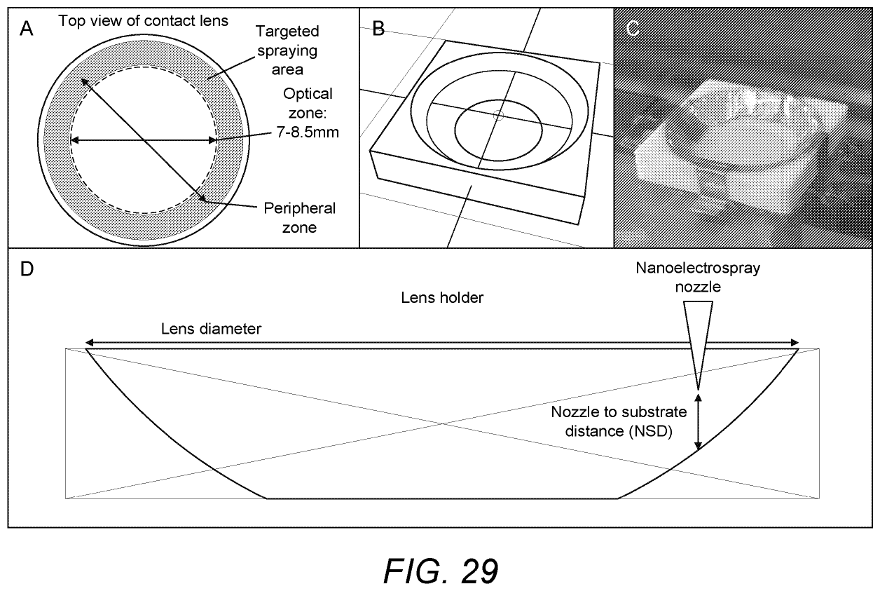

Commercial contact lenses were removed from the packaging and soaked in PBS pH 7.4 for 30 minutes to equilibrate. Excess liquid on the lens was removed by lint-free wipes (RS 558-795, RS Components, UK). 20 μL of PBS 7.4 was pipetted onto the 3D printed lens holder before fitting the semi-dry contact lens on the holder (FIG. 29). The drug-polymer solution was sprayed in the peripheral region (FIG. 29A) of the contact lens with the parameters as shown in Table 10 (the results shown are averages of three repeats of the experiments). Once the lenses were nanoelectrosprayed, they were stored in a container with lint-free cloth moistened with PBS to maintain hydration until the in vitro experiments.

TABLE 10
Summary of polymer-drug solutions used for
nES and the associated spraying parameters

| | KF1 | KF2 | KF3 | BIM1 |
|---|---|---|---|---|
| Zein (% w/v) | 5 | | | |
| Bimatoprost (%)* | 0 | 0 | 0 | 5 |
| Ketotifen fumarate (%)* | 15 | 5 | 1.5 | 0 |
| Conductivity (μS/cm), | | 371 | 319 | 291 |
| 25° C. | | | | |
| Surface tension ± SD | | 25.2 ± 0.8 | 25.6 ± 0.8 | 25.8 ± 0.7 |
| (mN/m) | | | | |
| Density ± SD (g/ml) | | 0.880 ± 0.001 | 0.8797 ± 0.002 | 0.880 ± 0.001 |
| Amount deposited on | 63.6 ± 1.93 | 26.85 ± 4.19 | 13.11 ± 1.78 | 30.81 ± 0.44 |
| lens (μg) | | | | |
| NSD (mm) | 2.34 | | | |
| Nozzle rotation speed | 15 | | | |
| Number of revolutions | 90 | | | |
| Voltage (kV) | 1.95 | 1.90 | 2.00 | 2.10 |

*the % is relative to polymer weight

### In Vitro Drug Release Tests

Glass vials containing 2 ml PBS 7.4 was incubated in a shaking incubator set at 35° C. with 125 rpm for 2 hours before in vitro experiments. All in vitro experiments were performed under sink conditions (i.e., the drug concentration within the in vitro dissolution medium was more than 10 times diluted in comparison to the saturated solubility of the drug in the medium). The nanoelectrosprayed lenses were placed into the vial and an aliquot of 1.5 ml was replaced with fresh PBS at regular intervals up to 24 hours (for KF the intervals were 0.5, 1, 3, 6, and 24 hours; for BIM the intervals were 0.25, 0.5, 0.75, 1, 2, 3, 4, 5, 6, and 24 hours). Any remaining drug left on the lens was extracted by PBS until no peak was observed in an HPLC chromatogram. The total amount of ketotifen and bimatoprost sprayed on a contact lens was determined by the sum of the cumulative amount of the drug released during the in vitro study and the amount obtained from extraction.

### High Performance Liquid Chromatography Instrumentation and Assay Methods

The high-performance liquid chromatography system (Jasco, Japan) consists of a pump (PU-1580), an autosampler (AS-2055 Plus) and a 4-channel UV detector (UV-1570M). A Waters C₁₈ column (250×4.6 mm i.d.; 5 μm particle size) was used under ambient conditions. The mobile phase of ketotifen fumarate [21] and bimatoprost [22] was prepared according to a method adopted from the literature. The mobile phase for ketotifen comprised methanol and 0.2% triethylamine (v/v) (80:20), running with 1 ml/min flow rate. The mobile phase for bimatoprost included acetonitrile, methanol and 0.1% phosphoric acid (v/v/v) (30:30:40), operating with a 1 ml/min flow rate. The injection volume was 20 μL and the detection wavelength was 300 nm and 210 nm for ketotifen and bimatoprost, respectively. Sample preparation of an aliquot was done to improve the peak intensity for both drug candidates. The aliquot collected from the in vitro experiment was mixed with the associated mobile phase in a 1:1 ratio, followed by filtration via a 0.2 μm (15141499, Fisher scientific, UK) syringe filter before the assay.

### Physiochemical Properties of the Solutions Used for nES

Conductivity: Conductivity measurements were performed by using a Jenway 4510 conductivity meter with a micro volume conductivity probe.

Surface tension: Surface tension of the solutions was measured by the pendant drop method (DMS-401, Kyowa interfacial science, Japan) For each sample, ten measurements were carried out to calculate the average surface tension.

Density: Density of the solutions was measured by a density meter (DMA 4500M, Anton Paar, Germany) equipped with an oscillating U-tube. The measurement was performed by injecting 1 ml of the sample into the system at 25° C. The measurement was taken in triplicate to obtain the average density.

Morphological Studies by Cryo-Scanning Electron Microscopy (cryoSEM)

Images of the surface and cross section of nanoelectrosprayed lenses were taken by using cryo-SEM (Zeiss, Gemini300, UK). Immediately after nanoelectrospraying, the lenses were cut in ⅛ of the whole lens and loaded onto an SEM stub for fast cooling with solid nitrogen. Samples were then prepared by platinum sputter coating before imaging.

### Results and Discussion

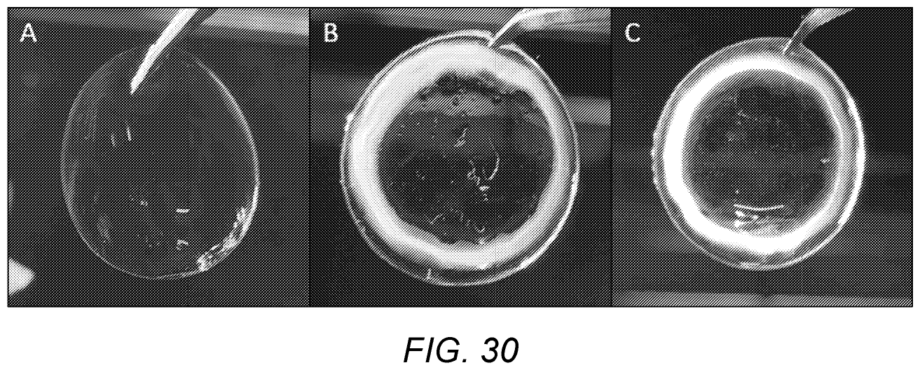

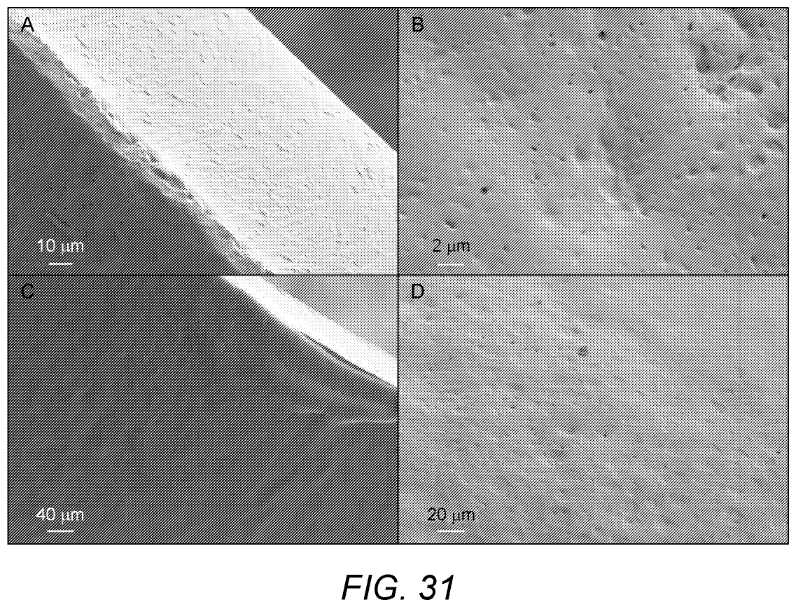

Table 10 summarises the physiochemical properties of the drug-polymer formulations used for nES. The digital images shown in FIG. 30 demonstrate the success of depositing zein-drug formulations in the peripheral area of a contact lens while keeping the centre of the lens clear. Cryo-SEM images shown in FIG. 31 show the surface morphology of the films with bimatoprost and ketotifen fumarate on a contact lens. It can be seen that a continuous film of both ketotifen and bimatoprost was successfully deposited onto the contact lens surface.

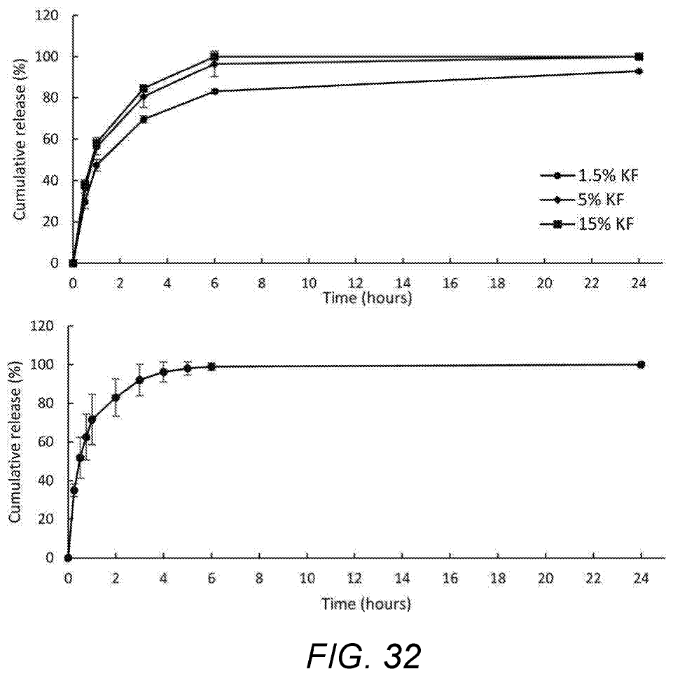

FIG. 32 (top) shows the in vitro drug release of ketotifen from a contact lens prepared by nES. A rapid release of the drug in the first 30 minutes (releasing 26% (1.5% w/w), 38% (5% w/w) and 37% (15% w/w)) was observed for all samples with different drug loadings. The release of ketotifen reached nearly 100% after 6 hours for a 15% w/w and 5% w/w drug loading and 93% after 24 hours for a 1.5% % w/w drug loading. The results indicate that for a water-soluble drug, such as ketotifen, the drug release kinetics are highly affected by the drug to polymer ratio in the film formulation.

According to the BNF (the British National Formulary), the strength of a ketotifen fumarate eye drop is 0.025% which is equivalent to 250 μg/mL. Taking the 5% bioavailability into consideration and the dose (1 drop twice daily), the required amount of ketotifen would be 1.25 μg per day [23]. The amount of ketotifen deposited onto a contact lens was higher than the therapeutic target in all cases. A further reduction in drug loading of ketotifen to 0.5% to obtain an even slower drug release with a sufficient dosage amount can be easily achieved to meet the therapeutic requirements.

FIG. 32 (bottom) shows the in vitro release of bimatoprost from contact lenses prepared by nanoelectrospraying. There was 30.81±0.44 μg of bimatoprost loaded onto the contact lens. According to the BNF, there are two strengths of bimatoprost available, 0.01% and 0.03%, which are equivalent to 100 or 300 μg/mL, respectively [24]. Taking the 5% bioavailability into consideration and the dose (1 drop per night), the required amount of bimatoprost would be 0.25 μg to 0.75 μg per day. The contact lenses prepared by nES carry a sufficient amount to meet the therapeutic dose. Rapid release of 51% of the bimatoprost in the first 30 minutes was observed. The drug release reached 98% after 6 hours. The low thickness of the zein film on the nanoelectrosprayed contact lenses could be the reason for quick the release of bimatoprost because of the short distance the drug molecules are required to travel to reach the surface. Therefore, adjusting the drug-polymer ratio and the thickness of the film could facilitate modulation of the drug release rate.

### REFERENCES MENTIONED HEREIN OR OTHERWISE USEFUL FOR BACKGROUND

1. Da Silva, G. R., et al., Ocular biocompatibility of dexamethasone acetate loaded poly(ε-caprolactone) nanofibers. European Journal of Pharmaceutics and Biopharmaceutics, 2019. 142: p. 20-30.
2. Jain, R. A., The manufacturing techniques of various drug loaded biodegradable poly(lactide-co-glycolide) (PLGA) devices. Biomaterials, 2000. 21(23): p. 2475-90.
3. Mir, M., N. Ahmed, and A. U. Rehman, Recent applications of PLGA based nanostructures in drug delivery. Colloids Surf B Biointerfaces, 2017. 159: p. 217-231.
4. Guo, X., et al., Inhalable microspheres embedding chitosan-coated PLGA nanoparticles for 2-methoxyestradiol. J Drug Target, 2014. 22(5): p. 421-7.
5. Gonzalez-Pizarro, R., et al., Development of fluorometholone-loaded PLGA nanoparticles for treatment of inflammatory disorders of anterior and posterior segments of the eye. International journal of pharmaceutics, 2018. 547(1-2): p. 338-346.
6. Berardi, A., et al., Zein as a Pharmaceutical Excipient in Oral Solid Dosage Forms: State of the Art and Future Perspectives. AAPS PharmSciTech, 2018. 19(5): p. 2009-2022.
7. Stuart, J. C. and J. G. Linn, Dilute sodium hyaluronate (Healon) in the treatment of ocular surface disorders. Ann Ophthalmol, 1985. 17(3): p. 190-2.
8. Aragona, P., et al., Long term treatment with sodium hyaluronate-containing artificial tears reduces ocular surface damage in patients with dry eye. Br J Ophthalmol, 2002. 86(2): p. 181-4.
9. Aragona, P., et al., Sodium hyaluronate eye drops of different osmolarity for the treatment of dry eye in Sjogren's syndrome patients. Br J Ophthalmol, 2002. 86(8): p. 879-84.
10. Hamano, T., et al., Sodium hyaluronate eyedrops enhance tear film stability. Jpn J Ophthalmol, 1996. 40(1): p. 62-5.
11. Sedlacek, J., [Possibility of the application of ophthalmic drugs with the use of gel contact lenses]. Cesk Oftalmol, 1965. 21(6): p. 509-12.
12. Xu, J., et al., A comprehensive review on contact lens for ophthalmic drug delivery. J Control Release, 2018. 281: p. 97-118.
13. Bengani, L. C., et al., Contact lenses as a platform for ocular drug delivery. Expert Opin Drug Deliv, 2013. 10(11): p. 1483-96.
14. Ciolino, J. B., et al., In vivo performance of a drug-eluting contact lens to treat glaucoma fora month. Biomaterials, 2014. 35(1): p. 432-9.
15. Mehta, P. et al. Development and characterisation of electrospun timolol maleate-loaded polymeric contact lens coatings containing various permeation enhancers. Int. J. Pharm. 2017, 532 (1) 408-420
16. Derby, B., Inkjet Printing of Functional and Structural Materials: Fluid Property Requirements, Feature Stability, and Resolution. Annual Review of Materials Research, 2010. 40(1): p. 395-414.
17. Cloupeau, M. and B. Prunet-Foch, Electrohydrodynamic spraying functioning modes: a critical review. Journal of Aerosol Science, 1994. 25(6): p. 1021-1036.
18. Fenn, J. B., et al., Electrospray ionization for mass spectrometry of large biomolecules. Science, 1989. 246(4926): p. 64-71.
19. Paine, M. D., et al., Controlled electrospray pulsation for deposition of femtoliter fluid droplets onto surfaces. Journal of Aerosol Science, 2007. 38(3): p. 315-324.
20. Alexander, M. S., M. D. Paine, and J. P. W. Stark, Pulsation Modes and the Effect of Applied Voltage on Current and Flow Rate in Nanoelectrospray. Analytical Chemistry, 2006. 78(8): p. 2658-2664.
21. Abd E I-Bary A A, Salem H F, Kharshoum R M. 2-Hydroxypropyl-β-cyclodextrin complex with ketotifen fumerate for eye drops preparations. Int J Drug Deliv. 2011; 1(2):228-40.
22. Franca J R, Batista L D, Ribeiro T G, Fernandes C, Castilho R O, Faraco A A G. Development and Validation of a High Performance Liquid Chromatographic Method for Determination of Bimatoprost in Chitosan-Based Ocular Inserts. Anal Lett. 2015; 48(3):531-40.
23. Ketotifen. BNF [Internet]. 2021; Available from: https://bnf.nice.org.uk/drug/ketotifen.html
24. Bimatoprost. BNF [Internet]. 2021; Available from: https://bnf.nice.org.uk/drug/bimatoprost.html#indicationsAndDoses

1. A method of coating an ocular prosthetic device, the method comprising
nanoelectrospraying droplets comprising an active ingredient and/or a carrier species onto a surface of the ocular prosthetic device in a predetermined pattern, the nanoelectrospraying involving controlling the flow rate of the droplets from a nozzle of the nanoelectrospraying equipment by controlling the voltage between the nozzle and the ocular prosthetic device.

2. The method according to claim 1, wherein the pattern covers only a portion of the surface of the ocular prosthetic device, and wherein a further portion of the ocular prosthetic device that, in use, would cover at least some of a pupil of an eye of a wearer of the ocular prosthetic device, is free of the predetermined pattern of droplets, and optionally the pattern comprises one or more features selected from dots, lines and regular shapes.

3. The method according to claim 1, wherein the droplets, and the active ingredient and/or carrier species, are coated on the ocular prosthetic device so that they form a ring, which may be continuous or discontinuous, around the periphery of the ocular prosthetic device, when viewed from above the ocular prosthetic device.

4. The method of claim 1, wherein the active ingredient and the carrier species are present in the droplets.

5. The method of claim 1, wherein the active ingredient is a pharmaceutically active ingredient for treating a disorder of the eye and the carrier species is a biologically compatible polymer.

6. The method of claim 5, wherein the pharmaceutically active ingredient is selected from ketotifen fumarate, timolol, latanoprost, or sodium hyaluronate.

7. The method of claim 5, wherein the biologically compatible polymer is selected from poly (lactic-co-glycolic acid), poly(lactide-co-caprolactone), zein, or sodium hyaluronate.

8. The method of claim 5, wherein the polymer is present in the droplets in a concentration of from 5 mg/ml to 75 mg/ml.

9. The method of claim 1, wherein the nozzle has an internal diameter of 250 μm or less.

10. The method of claim 1, wherein the nozzle has an internal diameter of 150 μm or less.

11. The method of claim 1, wherein the distance from the nozzle to the ocular prosthetic device during the nanoelectrospraying is from 0.5 mm to 3 mm.

12. The method of claim 1, wherein the voltage applied between the nozzle and the ocular prosthetic device to effect ejection of the droplets from the nozzle is from 1.5 kV to 3 kV.

13. The method of claim 1, wherein the ocular prosthetic device comprises a hydrogel material.

14. The method of claim 13, wherein the hydrogel material comprises water in an amount of from 10% to 80% by weight of the hydrogel material.

15. The method of claim 14, wherein the conductivity of the droplet, measured in a sample of a bulk of the liquid of the droplets, is from 5 μS/m to 100 μS/m.

16. An ocular prosthetic device formable according to the method of claim 1.

17. The ocular prosthetic device according to claim 16, wherein the device is a contact lens.

18. A method of coating a surface of a prosthetic device, the method comprising
nanoelectrospraying droplets comprising an active ingredient and/or a carrier species onto the surface in a predetermined pattern, the nanoelectrospraying involving controlling the flow rate of the droplets from a nozzle of the nanoelectrospraying equipment by controlling the voltage between the nozzle and the prosthetic device.

19. The method according to claim 18, wherein the surface is curved and/or electrically conductive.

20. The method according to claim 18, wherein the prosthetic device is selected from a stent, a heart valve, an artificial heart or any component thereof including power supply and regulator, a breast prosthesis, a larynx prosthesis, a trachea prosthesis, a tracheobronchial prosthesis, an ear or nose prosthesis, an internal organ prosthesis, biological tissue prosthesis, a limb prosthesis, hand prosthesis, or a foot prosthesis.
